(19) United States
(12) Patent Application Publication
(10) Pub. No.: US 2006/0263355 A1
(43) Pub. Date: Nov. 23, 2006

(54) Treatment of bone disorders

(76) Inventors: Joanne Quan, San Mateo, CA (US); K. Lea Sewell, San Francisco, CA (US)

Correspondence Address: SIDLEY AUSTIN LLP;ATTN: DC PATENT DOCKETING, 1501 K STREET, NW, WASHINGTON, DC, 20005, US

(21) Appl. No.: 11/363,091

(22) Filed: Feb. 28, 2006

Related U.S. Application Data
(60) Provisional application No. 60/656,943, filed on Feb. 28, 2005.

(51) Int. Cl. A61K 39/395; A61K 38/20; A61K 38/19; A61K 38/18; A61K 31/716; A61K 31/66; A61K 31/59

(52) U.S. Cl. 424/141.1; 424/143.1; 424/178.1; 424/85.1; 424/85.2; 514/9; 514/109; 514/167; 514/171; 514/54

(57) ABSTRACT

Methods of treatment of various bone indications, such as osteoporosis, in a mammal are provided wherein an effective amount of an antagonist that binds to a B-cell surface marker, such as a CD20 antibody, is administered, optionally also with another medicament such as an agent that treats such disorders in an effective amount. Articles of manufacture are also provided. Further, a method of inhibiting osteolysis in a mammal is provided comprising introducing into said mammal an isolated odontoprogenitor or osteoprogenitor cell comprising a nucleic acid encoding an antibody that binds to a B-cell surface marker.

This application claims priority under 35 U.S.C. § 119(e) from U.S. provisional patent application No. 60/656,943, filed 28 Feb. 2005, the entire contents of which are incorporated by reference.

### FIELD OF THE INVENTION

The present invention concerns treatment of bone disorders with antagonists that bind to B-cell surface markers, such as CD20 or CD22, e.g. antibodies that bind to CD20.

### BACKGROUND OF THE INVENTION

Bone Disorders

Bone remodeling is the dynamic process by which tissue mass and skeletal architecture are maintained. The process is a balance between bone resorption and bone formation, with two cell types, the osteoclast and osteoblast, thought to be the major players. Osteoblasts synthesize and deposit new bone into cavities that are excavated by osteoclasts. The activities of osteoblasts and osteoclasts are regulated by many factors, systemic and local, including growth factors.

Bone is a specialized dynamic connective tissue that serves the following functions: (a) mechanical, support and site of muscle attachment for locomotion; (b) protective, for vital organs and bone marrow: (c) metabolic, as reserve of ions, especially calcium and phosphate, for the maintenance of serum homeostasis, which is essential for life. Bone undergoes continuous resorption and renewal, a process collectively known as remodeling. Thus, the mechanical and biological integrity of bone dependents on its continuous destruction (resorption) and continuous rebuilding (formation) at millions of microscopic sites. During adult life bone remodeling is crucial to eliminate and replace structurally damaged or aged bone with structurally new healthy bone. To maintain the proper bone mass, resorption and formation are kept in perfect equilibrium. With age, the equilibrium between bone resorption and formation becomes altered, often in favor of resorption, resulting in a reduction in bone mass, deterioration of bone architecture, decreased resistance to stress, bone fragility and susceptibility to fractures. The compendium of these symptoms is referred to osteoporosis.

Osteoclasts are large multinuclear cells which function to erode bone matrix. They are related to macrophages and other cells that develop from monocytes. Like macrophages, osteoclasts are derived from haematopoietic progenitor cells. Osteoclasts mediate both systemic and focal bone loss.

Bone matrix erosion is a normal process that occurs in coordination with bone matrix formation, a process in which osteoblasts are involved. Essentially, osteoblasts erode bone matrix and tunnel into bone while osteoblasts follow, line the walls of the tunnel and form new bone matrix. Typically, in a normal adult, about 5-10% of bone is replaced by these processes annually.

Osteoporosis, a major health problem in Western society, is by far the most prevalent disease and is the most costly in terms of health care. The risk of osteoporosis is estimated to be 85% in women and 15% in men older than 45 years of age. In the United States it is estimated that 17 million post-menopausal women have lost 10% of their peak bone mass, 9.4 million have lost 25% and 5 million have suffered a fracture as a consequence of osteoporosis. Osteoporosis costs America's health care system more than $14 billion a year from spine and hip fractures, which are often the first indication of the disease if it is left undiagnosed.

Osteoporosis typically reflects an imbalance in skeletal turnover, such that bone resorption exceeds bone formation. Bone resorption is a specific function of osteoclasts, which are multinucleated, specialized bone cells formed by the fusion of mononuclear progenitors originating from the hemopoietic compartment, more precisely from the granulocyte-macrophage colony-forming unit (GM-CFU). The osteoclast is the principal cell type to resorb bone, and together with the bone-forming cells, the osteoblasts, dictate bone mass, bone shape and bone structure. The increased activity and/or numbers of osteoclasts, relative to the activity and or numbers of bone-forming osteoblasts, dictates the development of osteoporosis and other diseases of bone loss.

Even though Paget's disease is not as common or as costly as osteoporosis—it affects 3% of the population over 40, and 10% of the population over 80 years of age—it is nonetheless a significant disease because aside from causing bone fractures it can lead to severe osteoarthritis and severe neurological disorders. Paget's disease is characterized by rapid bone turnover, resulting in the formation of woven bone a tissue type formed initially in the embryo and during growth and which is practically absence from the adult skeleton. Woven bone is marked by brittleness and therefore prone to fractures and bowing. Bones become enlarged and often interfere with blood flow and constrict nerves, resulting in many of the neurological symptoms associated with Paget's disease.

For a disease in which osteoclasts presumably resorb bone at abnormally high levels and osteoblasts form bone at normal levels, as in osteoporosis, a reasonable therapeutic target would be the osteoclast: decreasing the number and/or the function of the osteoclasts may restore the equilibrium between bone resorption and formation. And, in fact, the treatments now available for osteoporosis are intended to suppress bone resorption.

Osteoclasts are derived from the monocyte-macrophage family. Upon stimulation of the CFU-GM with macrophage colony stimulating factor (M-CSF) form promonocytes which are immature nonadherent progenitors of mononuclear phagocytes and osteoclasts. The promonocytes may proliferate and differentiate along the macrophage pathway, eventually forming a tissue macrophage, or may differentiate along the osteoclast pathway, depending on the cytokines to which they become exposed. For example, the receptor activator NF-κB ligand (RANKL) (Simonet et al. Cell 89:309-319 (1997)), a cytokine expressed on the membrane surface of osteoblasts, influences promonocytes to differentiate into osteoclasts rather than macrophages, while treatment with M-CSF drives the promonocyte to develop into macrophages. Since M-CSF and other cytokines e.g., interleukin-1 or TNF-α, that support expression of RANKL are products of macrophages it may be assumed that immunomodulating substances, which alter the expression of, these cytokines and growth factors, may affect not only macrophages but also osteoclasts.

There are a number of therapeutic modalities for osteoporosis, which include bisphosphonates (Fleisch H, “Development of biphosphonates,” Breast Cancer Res. 4:30-34 (2002), Spencer, C P, Stevenson. J C “Oestrogen and anti-oestrogen for the prevention and treatment of osteoporosis.” In Osteoporosis: Diagnosis and Management, Martin Muniz, England, 1998, pp 111-123), or “Selective Estrogen Receptor Modulators” (SERMS).

Many of the proteins that influence the proliferation, differentiation, and activity of osteoblasts, osteoclasts, and their precursors also affect these processes in chondrocytes, the cells responsible for cartilage formation (chondrogenesis). These proteins include platelet-derived growth factor (PDGF), insulin-like growth factor (IGF), basic fibroblast growth factor (bFGF), transforming growth factor beta (TGF-β), bone morphogenetic proteins (BMPs), and cartilage-derived growth factor (CDGF).

A PDGF homolog known as “zvegf3” was recently identified and has also been designated “VEGF-R” (WIPO Publication WO 99/37671). Zvegf3/VEGF-R is a multi-domain protein with significant homology to the PDGF/VEGF family of growth factors. Zvegf3 was found to be useful in treating certain bone disorders such as osteoporosis (see US 2004/0043031 and U.S. Pat. No. 6,663,870).

A composition produced from living osteoprogenitor cells or odontoprogenitor cells is disclosed to deliver an anti-inflammatory polypeptide, for example, human interleukin-4, to build or rebuild bone tissue (see US 2004/0126364).

Receptors and ligands of the Tumor Necrosis Factor (TNF) family have recently been shown to play an essential part in the differentiation and activity of osteoclasts and therefore play a role in bone resorption. TNF-α is known to promote osteoclastogenesis, the generation of osteoclasts. A TNF-like molecule present on and/or secreted by osteoclasts and stromal cells, referred to interchangeably in the field and herein as “Receptor activator of NF-κ ligand”, (RANKL), “Osteoclast differentiation factor” (ODF), “Osteoprotegerin ligand” (OPGL), and “TNF-related activation-induced cytokine” (TRANCE), interacts with a TNF-receptor-like molecule, referred to in the field and herein as “Receptor activator of NF-κB ligand”, (RANK), which, present in the membranes of osteoclast precursors and mature osteoclasts, regulates osteoclastogenesis and the resorbing activity of mature osteoclasts. TNF inhibitors such as antibodies are used to treat rheumatoid arthritis. Suda et al. (Endocrine Reviews 20(3):345-357 (1999)) describe osteoclast differentiation and function. Filvaroff and Derynck (Curr. Biol. 8:R679-R682 (1998)) refer to bone remodeling and a signaling system for osteoclast regulation. See also Goldring, Journal of Musculoskeletal & Neuronal Interactions, 3(4): 287-289 (2003); Goldring, Rheumatology, 42 Suppl 2, pii1 1-6 (May 2003); Goldring and Goldring, Current Opinion in Orthopaedics, 13(5): 351-362 (2002); and Goldring, Current Opinion in Rheumatology 14(4) 406-10 (2002). Further, the arthritis that mice can develop is very inflammatory, but RANKL/ODF knockout mice do not get bone erosions (as reviewed by Gravallese, “Bone destruction in arthritis” Ann. Rheum. Dis. 61 (suppl II): ii84-6 (2002)).

US 2004/0058889 discloses methods of using beta-glucans to treat conditions associated with bone loss or low bone density as well as methods for promoting bone growth in situations where enhanced bone growth is desirable.

CD20 Antibodies and Therapy Therewith

Lymphocytes are one of many types of white blood cells produced in the bone marrow during the process of hematopoiesis. There are two major populations of lymphocytes: B lymphocytes (B cells) and T lymphocytes (T cells). The lymphocytes of particular interest herein are B cells.

B cells mature within the bone marrow and leave the marrow expressing an antigen-binding antibody on their cell surface. When a naïve B cell first encounters the antigen for which its membrane-bound antibody is specific, the cell begins to divide rapidly and its progeny differentiate into memory B cells and effector cells called “plasma cells”. Memory B cells have a longer life span and continue to express membrane-bound antibody with the same specificity as the original parent cell. Plasma cells do not produce membrane-bound antibody, but instead produce the antibody in a form that can be secreted. Secreted antibodies are the major effector molecules of humoral immunity.

The CD20 antigen (also called human B-lymphocyte-restricted differentiation antigen, Bp35) is a hydrophobic transmembrane protein with a molecular weight of approximately 35 kD located on pre-B and mature B lymphocytes. Valentine et al., J. Biol. Chem. 264(19):11282-11287 (1989) and Einfeld et al., EMBO J. 7(3):711-717 (1988). The antigen is also expressed on greater than 90% of B-cell non-Hodgkin's lymphomas (NHL) (Anderson et al. Blood 63(6):1424-1433 (1984)), but is not found on hematopoietic stem cells, pro-B cells, normal plasma cells, or other normal tissues (Tedder et al. J. Immunol. 135(2):973-979 (1985)). CD20 regulates an early step(s) in the activation process for cell-cycle initiation and differentiation (Tedder et al., supra), and possibly functions as a calcium-ion channel. Tedder et al., J. Cell. Biochem. 14D:195 (1990).

Given the expression of CD20 in B-cell lymphomas, this antigen can serve as a candidate for “targeting” of such lymphomas. In essence, such targeting can be generalized as follows: antibodies specific to the CD20 surface antigen of B cells are administered to a patient. These anti-CD20 antibodies specifically bind to the CD20 antigen of (ostensibly) both normal and malignant B cells; the antibody bound to the CD20 surface antigen may lead to the destruction and depletion of neoplastic B cells. Additionally, chemical agents or radioactive labels having the potential to destroy the tumor can be conjugated to the anti-CD20 antibody such that the agent is specifically “delivered” to the neoplastic B cells. Irrespective of the approach, a primary goal is to destroy the tumor; the specific approach can be determined by the particular anti-CD20 antibody that is utilized, and thus, the available approaches to targeting the CD20 antigen can vary considerably.

The rituximab antibody (the active agent in the products marketed in the U.S. as RITUXAN® and elsewhere as MABTHERA®) is a genetically engineered chimeric murine/human monoclonal antibody directed against the CD20 antigen. Rituximab is the antibody called “C2B8” in U.S. Pat. No. 5,736,137 issued Apr. 7, 1998 (Anderson et al.). Rituximab is indicated for the treatment of patients with relapsed or refractory low-grade or follicular, CD20-positive, B-cell non-Hodgkin's lymphoma. In vitro mechanism-of-action studies have demonstrated that rituximab binds human complement and lyses lymphoid B-cell lines through complement-dependent cytotoxicity (CDC). Reff et al., Blood 83(2):435-445 (1994). Additionally, it has significant activity in assays for antibody-dependent cellular cytotoxicity (ADCC). More recently, rituximab has been shown to have anti-proliferative effects in tritiated thymidine-incorporation assays and to induce apoptosis directly, while other anti-CD19 and anti-CD20 antibodies do not. Maloney et al. Blood 88(10):637a (1996). Synergy between rituximab and chemotherapies and toxins has also been observed experimentally. In particular, rituximab sensitizes drug-resistant human B-cell lymphoma cell lines to the cytotoxic effects of doxorubicin, CDDP, VP-16, diphtheria toxin, and ricin (Demidem et al., Cancer Chemotherapy & Radiopharmaceuticals 12(3):177-186 (1997)). In vivo preclinical studies have shown that rituximab depletes B cells from the peripheral blood, lymph nodes, and bone marrow of cynomolgus monkeys, presumably through complement- and cell-mediated processes. Reff et al., Blood 83:435-445 (1994).

Rituximab was approved in the United States in November 1997 for the treatment of patients with relapsed or refractory low-grade or follicular CD20+B-cell NHL at a dose of 375 mg/m² weekly for four doses. In April 2001, the Food and Drug Administration (FDA) approved additional indications for the treatment of low-grade NHL: re-treatment (weekly for four doses) and an additional dosing regimen (weekly for eight doses). There have been more than 300,000 patient exposures to rituximab either as monotherapy or in combination with immunosuppressant or chemotherapeutic drugs. Patients have also been treated with rituximab as maintenance therapy for up to 2 years. Hainsworth et al., J. Clin. Oncol. 21:1746-1751 (2003); Hainsworth et al., J. Clin. Oncol. 20:4261-4267 (2002); Edwards et al., “Efficacy of B-cell-targeted therapy with rituximab in patients with rheumatoid arthritis” N Engl. J. Med. 350:2572-82 (2004).

Rituximab has also been studied in a variety of non-malignant autoimmune disorders, in which B cells and autoantibodies appear to play a role in disease pathophysiology. Edwards et al., Biochem Soc. Trans. 30:824-828 (2002). Rituximab has been reported to potentially relieve signs and symptoms of, for example, rheumatoid arthritis (RA) (Leandro et al., Ann. Rheum. Dis. 61:883-888 (2002); Edwards et al., Arthritis Rheum., 46 (Suppl. 9): S46 (2002); Stahl et al., Ann. Rheum. Dis., 62 (Suppl. 1): OP004 (2003); Emery et al., Arthritis Rheum. 48(9): S439 (2003)), lupus (Eisenberg, Arthritis. Res. Ther. 5:157-159 (2003); Leandro et al. Arthritis Rheum. 46: 2673-2677 (2002); Gorman et al., Lupus, 13: 312-316 (2004)), immune thrombocytopenic purpura (D'Arena et al., Leuk. Lymphoma 44:561-562 (2003); Stasi et al., Blood, 98: 952-957 (2001); Saleh et al., Semin. Oncol., 27 (Supp 12):99-103 (2000); Zaia et al., Haematolgica, 87: 189-195 (2002); Ratanatharathorn et al., Ann. Int. Med., 133: 275-279 (2000)), pure red cell aplasia (Auner et al., Br. J. Haematol., 116: 725-728 (2002)); autoimmune anemia (Zaja et al., Haematologica 87:189-195 (2002) (erratum appears in Haematologica 87:336 (2002)), cold agglutinin disease (Layios et al. Leukemia, 15: 187-8 (2001); Berentsen et al., Blood, 103: 2925-2928 (2004); Berentsen et al., Br. J. Haematol., 115: 79-83 (2001); Bauduer, Br. J. Haematol., 112: 1083-1090 (2001); Damiani et al., Br. J. Haematol., 114: 229-234 (2001)), type B syndrome of severe insulin resistance (Coll et al., N. Engl. J. Med., 350: 310-311 (2004), mixed cryoglobulinemia (DeVita et al., Arthritis Rheum. 46 Suppl. 9:S206/S469 (2002)), myasthenia gravis (Zaja et al., Neurology, 55: 1062-63 (2000); Wylam et al., J. Pediatr., 143: 674-677 (2003)), Wegener's granulomatosis (Specks et al., Arthritis & Rheumatism 44: 2836-2840 (2001)), refractory pemphigus vulgaris (Dupuy et al., Arch Dermatol., 140:91-96 (2004)), dermatomyositis (Levine, Arthritis Rheum., 46 (Suppl. 9):S1299 (2002)), Sjogren's syndrome (Somer et al., Arthritis & Rheumatism, 49: 394-398 (2003)), active type-II mixed cryoglobulinemia (Zaja et al., Blood, 101: 3827-3834 (2003)), pemphigus vulgaris (Dupay et al. Arch. Dermatol., 140: 91-95 (2004)), autoimmune neuropathy (Pestronk et al., J. Neurol. Neurosurg. Psychiatry 74:485-489 (2003)), paraneoplastic opsoclonus-myoclonus syndrome (Pranzatelli et al. Neurology 60(Suppl. 1) PO5.128:A395 (2003)), and relapsing-remitting multiple sclerosis (RRMS). Cross et al. (abstract) “Preliminary Results from a Phase II Trial of Rituximab in MS” Eighth Annual Meeting of the Americas Committees for Research and Treatment in Multiple Sclerosis, 20-21 (2003).

A Phase II study (WA16291) has been conducted in patients with rheumatoid arthritis (RA), providing 48-week follow-up data on safety and efficacy of rituximab. Emery et al. Arthritis Rheum 48(9):S439 (2003); Szczepanski et al. Arthritis Rheum 48(9):S121 (2003). A total of 161 patients were evenly randomized to four treatment arms: methotrexate, rituximab alone, rituximab plus methotrexate, and rituximab plus cyclophosphamide (CTX). The treatment regimen of rituximab was one gram administered intravenously on days 1 and 15. Infusions of rituximab in most patients with RA were well tolerated by most patients, with 36% of patients experiencing at least one adverse event during their first infusion (compared with 30% of patients receiving placebo). Overall, the majority of adverse events was considered to be mild to moderate in severity and was well balanced across all treatment groups. There were a total of 19 serious adverse events across the four arms over the 48 weeks, which were slightly more frequent in the rituximab/CTX group. The incidence of infections was well balanced across all groups. The mean rate of serious infection in this RA patient population was 4.66 per 100 patient-years, which is lower than the rate of infections requiring hospital admission in RA patients (9.57 per 100 patient-years) reported in a community-based epidemiologic study. Doran et al., Arthritis Rheum. 46:2287-2293 (2002).

The reported safety profile of rituximab in a small number of patients with neurologic disorders, including autoimmune neuropathy (Pestronk et al., supra), opsoclonus-myoclonus syndrome (Pranzatelli et al., supra), and RRMS (Cross et al., supra), was similar to that reported in oncology or RA. In an ongoing investigator-sponsored trial (IST) of rituximab in combination with interferon-beta (IFN-β) or glatiramer acetate in patients with RRMS (Cross et al., supra), 1 of 10 treated patients was admitted to the hospital for overnight observation after experiencing moderate fever and rigors following the first infusion of rituximab, while the other 9 patients completed the four-infusion regimen without any reported adverse events.

Patents and patent publications concerning CD20 antibodies and CD20 binding molecules include U.S. Pat. Nos. 5,776,456, 5,736,137, 5,843,439, 6,399,061, and 6,682,734, as well as US 2002/0197255, US 2003/0021781, US 2003/0082172, US 2003/0095963, US 2003/0147885 (Anderson et al.); U.S. Pat. No. 6,455,043 and WO 2000/09160 (Grillo-López, A.); WO 2000/27428 (Grillo-López and White); WO 2000/27433 (Grillo-López and Leonard); WO 2000/44788 (Braslawsky et al.); WO 2001/10462 (Rastetter, W.); WO01/10461 (Rastetter and White); WO 2001/10460 (White and Grillo-López); US 2001/0018041, US 2003/0180292, WO 2001/34194 (Hanna and Hariharan); US 2002/0006404 and WO 2002/04021 (Hanna and Hariharan); US 2002/0012665 and WO 2001/74388 (Hanna, N.); US 2002/0058029 (Hanna, N.); US 2003/0103971 (Hariharan and Hanna); US 2002/0009444 and WO 2001/80884 (Grillo-López, A.); WO 2001/97858 (White, C.); US 2002/0128488 and WO 2002/34790 (Reff, M.); WO 2002/060955 (Braslawsky et al.); WO 2002/096948 (Braslawsky et al.); WO 2002/079255 (Reff and Davies); U.S. Pat. No. 6,171,586 and WO 1998/56418 (Lam et al.); WO 1998/58964 (Raju, S.); WO 1999/22764 (Raju, S.); WO 1999/51642, U.S. Pat. No. 6,194,551, U.S. Pat. No. 6,242,195, U.S. Pat. No. 6,528,624 and U.S. Pat. No. 6,538,124 (Idusogie et al.); WO 2000/42072 (Presta, L.); WO 2000/67796 (Curd et al.); WO 2001/03734 (Grillo-López et al.); US 2002/0004587 and WO 2001/77342 (Miller and Presta); US 2002/0197256 (Grewal, I.); US 2003/0157108 (Presta, L.); U.S. Pat. Nos. 6,565,827, 6,090,365, 6,287,537, 6,015,542, 5,843,398, and 5,595,721, (Kaminski et al.); U.S. Pat. Nos. 5,500,362, 5,677,180, 5,721,108, 6,120,767, and 6,652,852 (Robinson et al.); U.S. Pat. No. 6,410,391 (Raubitschek et al.); U.S. Pat. No. 6,224,866 and WO00/20864 (Barbera-Guillem, E.); WO 2001/13945 (Barbera-Guillem, E.); WO 2000/67795 (Goldenberg); US 2003/0133930 and WO 2000/74718 (Goldenberg and Hansen); US 2003/0219433 and WO 2003/68821 (Hansen et al.); WO2004/058298 (Goldenberg and Hansen); WO 2000/76542 (Golay et al.); WO 2001/72333 (Wolin and Rosenblatt); U.S. Pat. No. 6,368,596 (Ghetie et al.); U.S. Pat. No. 6,306,393 and US 2002/0041847 (Goldenberg, D.); US 2003/0026801 (Weiner and Hartmann); WO 2002/102312 (Engleman, E.); US 2003/0068664 (Albitar et al.); WO 2003/002607 (Leung, S.); WO 2003/049694, US2002/0009427, and US 2003/0185796 (Wolin et al.); WO 2003/061694 (Sing and Siegall); US 2003/0219818 (Bohen et al.); US 2003/0219433 and WO 2003/068821 (Hansen et al.); US 2003/0219818 (Bohen et al.); US2002/0136719 (Shenoy et al.); WO 2004/032828 (Wahl et al.); and WO 2002/56910 (Hayden-Ledbetter). See also U.S. Pat. No. 5,849,898 and EP 330,191 (Seed et al.); EP332,865A2 (Meyer and Weiss); U.S. Pat. No. 4,861,579 (Meyer et al.); US2001/0056066 (Bugelski et al.); WO 1995/03770 (Bhat et al.); US 2003/0219433 A1 (Hansen et al.); WO 2004/035607 (Teeling et al.); WO 2004/056312 (Lowman et al.); US 2004/0093621 (Shitara et al.); WO 2004/103404 (Watkins et al.); WO 2005/000901 (Tedder et al.); and US 2005/0025764 (Watkins et al.).

Publications concerning therapy with rituximab include: Perotta and Abuel, “Response of chronic relapsing ITP of 10 years duration to rituximab” Abstract # 3360 Blood 10(1)(part 1-2): p. 88B (1998); Perotta et al., “Rituxan in the treatment of chronic idiopathic thrombocytopaenic purpura (ITP)”, Blood, 94: 49 (abstract) (1999); Matthews, R., “Medical Heretics” New Scientist (7 Apr., 2001); Leandro et al., “Clinical outcome in 22 patients with rheumatoid arthritis treated with B lymphocyte depletion” Ann Rheum Dis, supra; Leandro et al., “Lymphocyte depletion in rheumatoid arthritis: early evidence for safety, efficacy and dose response” Arthritis and Rheumatism 44(9): S370 (2001); Leandro et al., “An open study of B lymphocyte depletion in systemic lupus erythematosus”, Arthritis and Rheumatism, 46:2673-2677 (2002), wherein during a 2-week period, each patient received two 500-mg infusions of rituximab, two 750-mg infusions of cyclophosphamide, and high-dose oral corticosieroids, and wherein two of the patients treated relapsed at 7 and 8 months, respectively, and have been retreated, although with different protocols; “Successful long-term treatment of systemic lupus erythematosus with rituximab maintenance therapy” Weide et al., Lupus, 12: 779-782 (2003), wherein a patient was treated with rituximab (375 mg/m²×4, repeated at weekly intervals) and further rituximab applications were delivered every 5-6 months and then maintenance therapy was received with rituximab 375 mg/m² every three months, and a second patient with refractory SLE was treated successfully with rituximab and is receiving maintenance therapy every three months, with both patients responding well to rituximab therapy; Edwards and Cambridge, “Sustained improvement in rheumatoid arthritis following a protocol designed to deplete B lymphocytes' Rheumatology 40:205-211 (2001); Cambridge et al., “B lymphocyte depletion in patients with rheumatoid arthritis: serial studies of immunological parameters” Arthritis Rheum., 46 (Suppl. 9): S1350 (2002); Edwards et al., “B-lymphocyte depletion therapy in rheumatoid arthritis and other autoimmune disorders” Biochem Soc. Trans., supra; Edwards et al., Efficacy and safety of rituximab, a B-cell targeted chimeric monoclonal antibody: A randomized, placebo controlled trial in patients with rheumatoid arthritis. Arthritis and Rheumatism 46(9): S197 (2002); Edwards et al., “Efficacy of B-cell-targeted therapy with rituximab in patients with rheumatoid arthritis,” N. Engl. J. Med. 350:2572-82 (2004); Pavelka et al., Ann. Rheum. Dis. 63: (S1):289-90 (2004); Emery et al., Arthritis Rheum. 50 (S9):S659 (2004); Levine and Pestronk, “IgM antibody-related polyneuropathies: B-cell depletion chemotherapy using rituximab” Neurology 52: 1701-1704 (1999); De Vita et al., “Efficacy of selective B cell blockade in the treatment of rheumatoid arthritis” Arthritis & Rheum 46:2029-2033 (2002); Hidashida et al. “Treatment of DMARD-refractory rheumatoid arthritis with rituximab.” Presented at the Annual Scientific Meeting of the American College of Rheumatology; October 24-29; New Orleans, La. 2002; Tuscano, J. “Successful treatment of infliximab-refractory rheumatoid arthritis with rituximab” Presented at the Annual Scientific Meeting of the American College of Rheumatology; October 24-29; New Orleans, La. 2002; “Pathogenic roles of B cells in human autoimmunity; insights from the clinic” Martin and Chan, Immunity 20:517-527 (2004); Silverman and Weisman, “Rituximab Therapy and Autoimmune Disorders, Prospects for Anti-B Cell Therapy”, Arthritis and Rheumatism, 48: 1484-1492 (2003); Kazkaz and Isenberg, “Anti B cell therapy (rituximab) in the treatment of autoimmune diseases”, Current opinion in pharmacology, 4: 398-402 (2004); Virgolini and Vanda, “Rituximab in autoimmune diseases”, Biomedicine & pharmacotherapy, 58: 299-309(2004); Klemmer et al. “Treatment of antibody mediated autoimmune disorders with a AntiCD20 monoclonal antibody Rituximab”, Arthritis And Rheumatism, 48: (9) 9,S (SEP), page: S624-S624(2003); Kneitz et al., “Effective B cell depletion with rituximab in the treatment of autoimmune diseases”, Immunobiology, 206: 519-527 (2002); Arzoo et al., “Treatment of refractory antibody mediated autoimmune disorders with an anti-CD20 monoclonal antibody (rituximab)” Annals of the Rheumatic Diseases, 61 (10), p922-4 (2002) Comment in Ann Rheum Dis. 61: 863-866 (2002); “Future Strategies in Immunotherapy” by Lake and Dionne, in Burger's Medicinal Chemistry and Drug Discovery (2003 by John Wiley & Sons, Inc.) Article Online Posting Date: Jan. 15, 2003 (Chapter 2“Antibody-Directed Immunotherapy”); Liang and Tedder, Wiley Encyclopedia of Molecular Medicine, Section: CD20 as an Immunotherapy Target, article online posting date: 15 Jan. 2002 entitled “CD20”: Appendix 4A entitled “Monoclonal Antibodies to Human Cell Surface Antigens” by Stockinger et al., eds: Coligan et al., in Current Protocols in Immunology (2003 John Wiley & Sons, Inc) Online Posting Date: May, 2003; Print Publication Date: February, 2003; Penichet and Morrison, “CD Antibodies/molecules: Definition; Antibody Engineering” in Wiley Encyclopedia of Molecular Medicine Section: Chimeric, Humanized and Human Antibodies; posted online 15 Jan., 2002; Specks et al. ‘Response of Wegener’s granulomatosis to anti-CD20 chimeric monoclonal antibody therapy” Arthritis & Rheumatism 44:2836-2840 (2001); online abstract submission and invitation Koegh et al., “Rituximab for Remission Induction in Severe ANCA-Associated Vasculitis: Report of a Prospective Open-Label Pilot Trial in 10 Patients”, American College of Rheumatology, Session Number: 28-100, Session Title: Vasculitis, Session Type: ACR Concurrent Session, Primary Category: 28 Vasculitis, Session Oct. 18, 2004 (<www.abstractsonline.com/viewer/SearchResults.asp>); Eriksson, “Short-term outcome and safety in 5 patients with ANCA-positive vasculitis treated with rituximab”, Kidney and Blood Pressure Research, 26: 294 (2003); Jayne et al., “B-cell depletion with rituximab for refractory vasculitis” Kidney and Blood Pressure Research, 26: 294 (2003); Jayne, poster 88 (11^{th} International Vasculitis and ANCA workshop), 2003 American Society of Nephrology; Stone and Specks, “Rituximab Therapy for the Induction of Remission and Tolerance in ANCA-associated Vasculitis”, in the Clinical Trial Research Summary of the 2002-2003 Immune Tolerance Network, <www.immunetolerance.org/research/autoimmune/trials/stone.html>. See also Leandro et al., “B cell repopulation occurs mainly from naïve B cells in patient with rheumatoid arthritis and systemic lupus erythematosus” Arthritis Rheum., 48 (Suppl 9): S1160 (2003).

The role of B lymphocytes in osteoclast formation is controversial, because both stimulatory and inhibitory effects of B-lineage cells on osteoclastogenesis and life span have been reported. B-lymphoid lineage cells may participate in osteoclastogenesis in two ways, by expressing ODF/RANKL to support osteoclast differentiation and to serve as osteoclast progenitors (Manabe et al., J. Immunol., 167 (5):2625-2631 (2001)). Osteoprotegerin regulates B-cell maturation as well as bone metabolism (Yun et al., J. Immunol., 166 (3): 1482-1491 (2001)). B cells have been shown to inhibit the formation of osteoclast formations and shorten the life span of mature osteoclast formations by secreting transforming growth factor-beta (TGF-β), a factor that induces apoptosis in these cells (Weitzmann et al., J. Cell. Biochem., 78: (2): 318-324 (2000)). B-lymphocyte progenitors may give rise to functional osteoclasts (Grcevic et al., Croatian Medical Journal, 42 (4): 384-392 (2001)).

The bisphosphonates in rat adjuvant arthritis studies include that etridronate and clodronate decrease weight loss, pedal inflammation, and bone resorption (Flora et al., Arthritis Rheum., 22:340-346 (1978)). NE58095 is a diphosphonate that prevents bone erosion and preserves joint architecture in experimental arthritis (Francis et al., Int. J. Tis. Res., 11:239-252 (1989)). Zoledronate protects against meta hyseal intracortical defets in experimental inflammatory arthritis (Pysklwec et al., J. Orthop. Res., 15: 858-861 (1997)). Clodronate 20 mg/kg/day iv infusion decreases pedal inflammation and bone resorption, but also inhibits bone formation. Oelzner et al., Inflamm. Res., 49: 424-433 (2000).

All approved bisphosphonates (pamidronate, alendronate, risedronate, zoledronate) improve biochemical measures of bone resorption (reduce systemic bone loss), as well as inhibit progression of focal bone erosions. Gravallese, supra. Although bisphosphonates might be considered as potential therapy to slow structural change in rheumatoid arthritis, the efficacy, long-term tolerability, and toxicity are unknown, with more trials needed.

Despite the increasing knowledge of the role of growth factors in tissue growth and repair, there remains a need in the art for materials and methods for promoting the growth of bone, ligament, and cartilage. There is also a need for methods of regulating osteoclastogenesis and the resorbing activity of mature osteoclasts. There is also a need for methods of preventing bone loss and treating bone diseases.

### SUMMARY OF THE INVENTION

Accordingly, the invention is as claimed. The present invention involves administration of an antagonist that binds to a B-cell surface marker that provides a safe and active treatment regimen in subjects with bone disorders.

In a first aspect, the present invention concerns a method for treating a bone disorder in a mammal comprising administering to the mammal an effective amount of a CD20 antibody.

In another aspect, the invention relates to a method for treating a bone disorder in a mammal comprising administering to the mammal an effective amount of an antibody that binds to a B-cell surface marker.

In still another aspect, the invention provides a method for treating a bone disorder in a mammal comprising administering to the mammal an effective amount of an antagonist that binds to a B-cell surface marker.

In a further aspect, the invention provides an article of manufacture comprising: a container comprising a CD20 antibody, or an antibody or antagonist that binds to a B-cell surface marker; and a package insert with instructions for treating a bone disorder in a mammal, wherein the instructions indicate that the CD20 antibody, or the antibody or antagonist that binds to a B-cell surface marker is administered to the mammal in an effective amount. In a preferred embodiment, the article further comprises a container comprising an agent other than the antibody for the treatment and further comprises instructions on treating the mammal with such agent.

In preferred embodiments of the above inventive aspects, the bone disorder is osteoporosis, or a focal bone erosion including marginal joint erosions and subchondral bone erosions (bone marrow), a bone defect, childhood idiopathic bone loss, alveolar bone loss, bone fracture, or osteopenia such as juxta-articular osteopenia, or is bone loss associated with an autoimmune disease such as rheumatoid arthritis. In a still further embodiment, the invention provides a method for preventing erosive bone disease in inflammatory arthritides, such as rheumatoid arthritis, comprising administering to a mammal suffering from such disease an effective amount of a CD20 antibody. Also within the invention are methods of treating bone disorders that are not associated with an autoimmune disease (in particular, rheumatoid arthritis) or a risk of developing an autoimmune disease.

In another preferred aspect, a second medicament is administered in an effective amount to the mammal, wherein the CD20 antibody, or the antibody or antagonist that binds to a B-cell surface marker, is a first medicament. Such medicament may be one or more medicaments. More preferably, such second medicament is an agent that treats osteoclast-associated disorders (such as osteoprotegerin, a MMP inhibitor, a cytokine such as IL-4, a beta glucan, an integrin antagonist, calcitonin, a proton pump inhibitor, a protease inhibitor, or a bisphosphonate such as risedronate, etidronate, clodronate, NE-58095, zoledronate, pamidronate, or alendronate), an immunosuppressive agent, a disease-modifying anti-rheumatic drug (DMARD), a cytotoxic agent, a non-steroidal anti-inflammatory drug (NSAID), a hormone, or a combination thereof. In yet another preferred embodiment, the mammal is human. The antibody is preferably administered intravenously or subcutaneously.

In still another preferred embodiment of these lattermost aspects, the mammal has never been previously treated with an antibody that binds to a B-cell surface marker, such as CD20 antibody, and/or no other medicament than the antibody that binds to a B-cell surface marker, including CD20 antibody, is administered to the mammal to treat the bone disorder.

In further aspects, the invention provides a method of inhibiting osteolysis in a mammal, comprising introducing into said mammal an isolated odontoprogenitor or osteoprogenitor cell comprising a nucleic acid encoding an antibody that binds to a B-cell surface marker, preferably a CD20 antibody. If the cell is an odontoprogenitor cell, said mammal may be suffering from or at risk of developing periodontitis, or alveolar bone loss due to periodontal disease. In another embodiment, the odontoprogenitor cell may be administered to the periodontal ligament in the mandibular section of the jaw.

In another embodiment, if the cell is an osteoprogenitor cell, it may be implanted into an articulating joint of said mammal, and may be administered intratibially or intrafemorally. Further, expression of said antibody may be regulated by an antibiotic compound, such as tetracycline or a tetracycline analogue, and further minocycline may be administered to said mammal. Such antibiotic compound is preferably administered systemically.

Interleukin-4 or an inhibitor of tumor necrosis factor-alpha (TNF-α) may be further administered to the mammal in such situation. Further, such mammal may be suffering from or at risk of developing rheumatoid arthritis or periapical or endochondral bone loss, artificial joint particle-induced osteolysis, or osteolytic bone metastases.

Conditions wherein promotion of bone growth is beneficial include, for example, strengthening a bone graft, inducing vertebral synostosis, enhancing long bone extension, enhancing bone healing following facial reconstruction, maxillary reconstruction and/or mandibular reconstruction in a vertebrate, e.g., a mammal (including a human being), and the like.

The inhibition of osteoclast activity achieved by the methods of this invention, without being limited to any one theory, can be the result of an inhibitory activity of the resorption mechanisms of the osteoclasts, or can be the result of an inhibition of the number of osteoclasts recruited from precursor cells, or a combination of both. In osteoporosis, which affects mostly older individuals and particularly post-menopausal women, bone formation by osteoblasts slows down and bone resorption increases, phenomena that occur normally due to the aging process. The method herein is intended, inter alia, to enhance osteoblast formation, thus also increasing bone formation.

### BRIEF DESCRIPTION OF THE DRAWINGS

FIG. 1A is a sequence alignment comparing the amino acid sequences of the light-chain variable domain (V_{L}) of each of murine 2H7 (SEQ ID NO:1), humanized 2H7.v16 variant (SEQ ID NO:2), and the human kappa light-chain subgroup 1 (SEQ ID NO:3). The CDRs of V_{L} of 2H7 and hu2H7.v16 are as follows: CDR1 (SEQ ID NO:4), CDR2 (SEQ ID NO:5), and CDR3 (SEQ ID NO:6).

FIG. 1B is a sequence alignment comparing the amino acid sequences of the heavy-chain variable domain (V_{H}) of each of murine 2H7 (SEQ ID NO:7), humanized 2H7.v16 variant (SEQ ID NO:8), and the human consensus sequence of the heavy-chain subgroup III (SEQ ID NO:9). The CDRs of V_{H} of 2H7 and hu2H7.v16 are as follows: CDR1 (SEQ ID NO:10), CDR2 (SEQ ID NO:11), and CDR3 (SEQ ID NO:12).

In FIG. 1A and FIG. 1B, the CDR1, CDR2 and CDR3 in each chain are boxed, flanked by the framework regions, FR1-FR4, as indicated. 2H7 refers to the murine 2H7 antibody. The asterisks in between two rows of sequences indicate the positions that are different between the two sequences. Residue numbering is according to Kabat et al. Sequences of Immunological Interest. 5th Ed. Public Health Service, National Institutes of Health, Bethesda, Md. (1991), with insertions shown as a, b, c, d, and e.

FIG. 2 is a sequence alignment comparing the light-chain amino acid sequences of the humanized 2H7.v16 variant (SEQ ID NO:2) and humanized 2H7.v138 variant (SEQ ID NO:28).

FIG. 3 is a sequence alignment comparing the heavy-chain amino acid sequences of the humanized 2H7.v16 variant (SEQ ID NO:8) and humanized 2H7.v138 variant (SEQ ID NO:29).

### DETAILED DESCRIPTION OF THE PREFERRED EMBODIMENTS

I. Definitions

A “B cell” is a lymphocyte that matures within the bone marrow, and includes a naçve B cell, memory B cell, or effector B cell (plasma cells). The B cell herein may be a normal or non-malignant B cell.

A “B-cell surface marker” or “B-cell surface antigen” herein is an antigen expressed on the surface of a B cell that can be targeted with an antagonist that binds thereto. Exemplary B-cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85 and CD86 leukocyte surface markers (for descriptions, see The Leukocyte Antigen Facts Book, 2^{nd} Edition. 1997, ed. Barclay et al. Academic Press, Harcourt Brace & Co., New York). Other B-cell surface markers include RP105, FcRH2, B-cell CR2, CCR6, P2X5, HLA-DOB, CXCR5, FCER2, BR3, Btig, NAG14, SLGC16270, FcRH1, IRTA2. ATWD578, FcRH3, IRTA1, FcRH6, BCMA, and 239287. The B-cell surface marker of particular interest is preferentially expressed on B cells compared to other non-B-cell tissues of a mammal and may be expressed on both precursor B cells and mature B cells. The preferred B-cell surface markers herein are CD20 and CD22.

The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35”. The CD20 antigen is described in Clark et al., Proc. Natl. Acad. Sci. (USA) 82:1766 (1985), for example.

The “CD22” antigen, or “CD22,” also known as BL-CAM or Lyb8, is a type I integral membrane glycoprotein with molecular weight of about 130 (reduced) to 140 kD (unreduced). It is expressed in both the cytoplasm and cell membrane of B-lymphocytes. CD22 antigen appears early in B-cell lymphocyte differentiation at approximately the same stage as the CD19 antigen. Unlike other B-cell markers, CD22 membrane expression is limited to the late differentiation stages comprised between mature B cells (CD22+) and plasma cells (CD22−). The CD22 antigen is described, for example, in Wilson et al., J. Exp. Med. 173:137 (1991) and Wilson et al., J. Immunol. 150:5013 (1993).

An “antagonist” is a molecule that, upon binding to CD20 on B cells, destroys or depletes B cells in a mammal and/or interferes with one or more B cell functions, e.g. by reducing or preventing a humoral response elicited by the B cell. The antagonist preferably is able to deplete B cells (i.e. reduce circulating B cell levels) in a mammal treated therewith. Such depletion may be achieved via various mechanisms such antibody-dependent cell-mediated cytotoxicity (ADCC) and/or complement dependent cytotoxicity (CDC), inhibition of B cell proliferation and/or induction of B cell death (e.g. via apoptosis). Antagonists included within the scope of the present invention include antibodies, synthetic or native-sequence peptides, immunoadhesins, and small-molecule antagonists that bind to CD20, optionally conjugated with or fused to a cytotoxic agent. The preferred antagonist comprises an antibody.

An “antibody antagonist” herein is an antibody that, upon binding to a B-cell surface marker on B cells, destroys or depletes B cells in a mammal and/or interferes with one or more B-cell functions, e.g., by reducing or preventing a humoral response elicited by the B cell. The antibody antagonist preferably is able to deplete B cells (i.e., reduce circulating B-cell levels) in a mammal treated therewith. Such depletion may be achieved via various mechanisms such antibody-dependent cell-mediated cytotoxicity (ADCC) and/or complement-dependent cytotoxicity (CDC), inhibition of B-cell proliferation and/or induction of B-cell death (e.g., via apoptosis).

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are included in this definition and described in WO00/42072 and US2005/0014934A1. These antibodies comprise an Fc region with one or more substitutions therein which improve binding of the Fc region to FcRn. The preferred Fc region-comprising antibody variant with improved FcRn binding comprises amino acid substitutions at one, two or three positions of the Fc region thereof.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

For the purposes herein, an “intact antibody” is one comprising heavy and light variable domains as well as an Fc region.

An “antibody that binds to a B-cell surface marker” is a molecule that, upon binding to a B-cell surface marker, destroys or depletes B cells in a mammal and/or interferes with one or more B-cell functions, e.g. by reducing or preventing a humoral response elicited by the B cell. The antibody preferably is able to deplete B cells (i.e. reduce circulating B-cell levels) in a mammal treated therewith. Such depletion may be achieved via various mechanisms such antibody-dependent cell-mediated cytotoxicity (ADCC) and/or complement-dependent cytotoxicity (CDC), inhibition of B-cell proliferation and/or induction of B-cell death (e.g. via apoptosis). In one preferred embodiment, the antibody induces a major clinical response. In another preferred embodiment, the B-cell surface marker is CD20 or CD22, so that the antibody that binds to a B-cell surface marker is an antibody that binds to CD20 or CD22, respectively, or a “CD20 antibody” or “CD22 antibody,” respectively. Examples of CD22 antibodies include the ones described in EP 1,476,120 (Tedder and Tuscano), EP 1,485,130 (Tedder), and EP 1,504,035 (Popplewell et al.), as well as those described in US 2004/0258682 (Leung et al.). In a still more preferred embodiment, the antibody is a CD20 antibody. A particularly preferred embodiment is a CD20 or CD22 antibody, preferably a CD20 antibody, that induces a major clinical response. For purposes herein, a “major clinical response” is defined as achieving an American College of Rheumatology 70 response (ACR 70) for six consecutive months. ACR response scores are categorized as ACR 20, ACR 50 and ACR 70 with ACR 70 being the highest level of sign and symptom control in this evaluation system. ACR response scores measure improvement in rheumatoid arthritis disease activity, including joint swelling and tenderness, pain, level of disability and overall patient and physician assessment. An example of a different type of antibody that induces a major clinical response as recognized by the FDA and as defined herein is etanercept (ENBREL®).

Examples of CD20 antibodies include: “C2B8,” which is now called “rituximab” (RITUXAN®/MABTHERA®(U.S. Pat. No. 5,736,137); the yttrium-[90]-labelled 2B8 murine antibody designated “Y2B8” or “Ibritumomab Tiuxetan” (ZEVALIN®) commercially available from Biogen Idec Inc. (U.S. Pat. No. 5,736,137; 2B8 deposited with ATCC under accession no. HB11388 on Jun. 22, 1993); murine IgG2a “B1,” also called “Tositumomab,” optionally labelled with ¹³¹I to generate the “131I-B1” or “iodine I131 tositumomab” antibody (BEXXAR™) commercially available from Corixa (see, also, U.S. Pat. No. 5,595,721); murine monoclonal antibody “1F5” (Press et al. Blood 69(2):584-591 (1987) and variants thereof including “framework patched” or humanized 1F5 (WO 2003/002607, Leung, S.; ATCC deposit HB-96450); murine 2H7 and chimeric 2H7 antibody (U.S. Pat. No. 5,677,180); a humanized 2H7 (WO 2004/056312 (Lowman et al.) and as set forth below); HUMAX-CD20™ fully human, high-affinity antibody targeted at the CD20 molecule in the cell membrane of B-cells (Genmab, Denmark; see, for example, Glennie and van de Winkel, Drug Discovery Today 8: 503-510 (2003) and Cragg et al., Blood 101: 1045-1052 (2003)); the human monoclonal antibodies set forth in WO 2004/035607 (Teeling et al.); the antibodies having complex N-glycoside-linked sugar chains bound to the Fc region described in US 2004/0093621 (Shitara et al.); monoclonal antibodies and antigen-binding fragments binding to CD20 (WO 2005/000901, Tedder et al.) such as HB20-3, HB20-4, HB20-25, and MB20-11; CD20 binding molecules such as the AME series of antibodies, e.g., AME-33™ antibodies as set forth in WO 2004/103404 (Watkins et al., Applied Molecular Evolution); CD20 binding molecules such as those described in US 2005/0025764 (Watkins et al.); A20 antibody or variants thereof such as chimeric or humanized A20 antibody (cA20, hA20, respectively) (US 2003/0219433, Immunomedics); and monoclonal antibodies L27, G28-2, 93-1B3, B-C1 or NU-B2 available from the International Leukocyte Typing Workshop (Valentine et al., In: Leukocyle Typing III (McMichael, Ed., p. 440, Oxford University Press (1987)). The preferred CD20 antibodies herein are chimeric, humanized, or human CD20 antibodies, more preferably rituximab, a humanized 2H7, chimeric or humanized A20 antibody (Immunomedics), and HUMAX-CD20™ human CD20 antibody (Genmab).

The terms “rituximab” or “RITUXAN®” herein refer to the genetically engineered chimeric murine/human monoclonal antibody directed against the CD20 antigen and designated “C2B8” in U.S. Pat. No. 5,736,137, including fragments thereof which retain the ability to bind CD20.

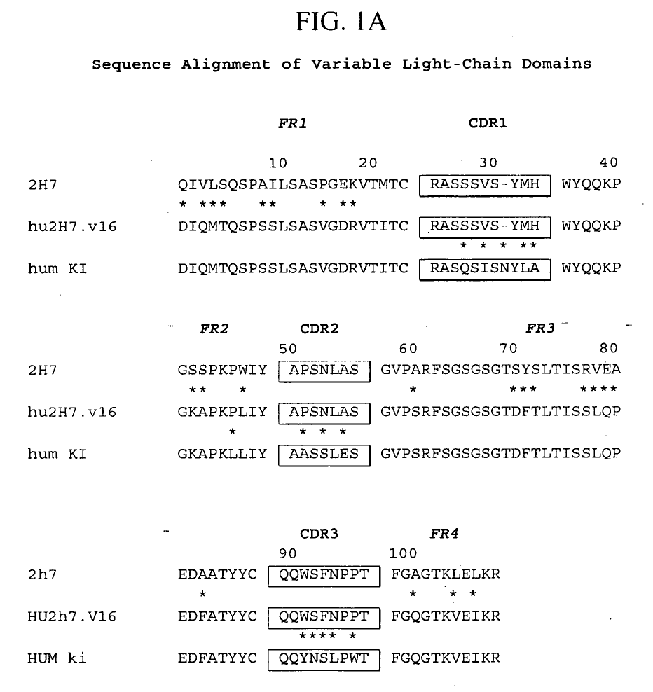

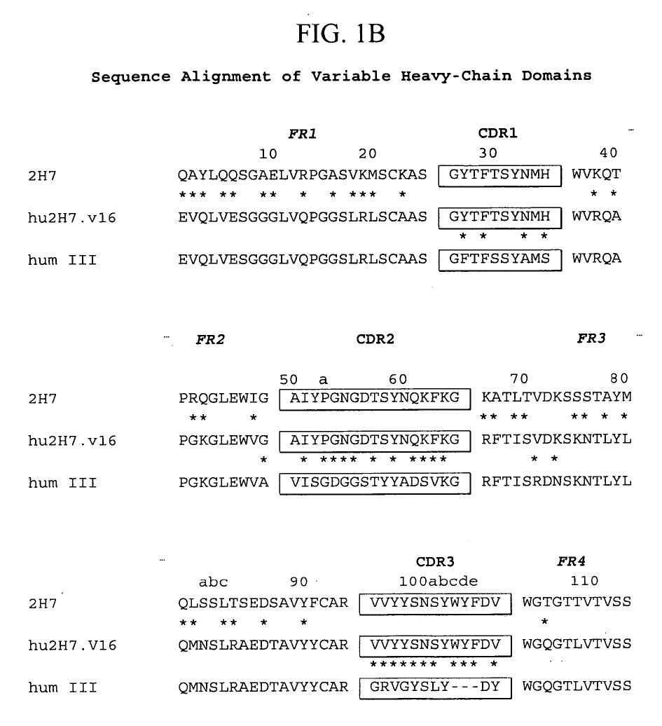

Purely for the purposes herein and unless indicated otherwise, a “humanized 2H7” refers to a humanized CD20 antibody, or an antigen-binding fragment thereof, wherein the antibody is effective to deplete primate B cells in vivo, the antibody comprising in the H chain variable region (V_{H}) thereof at least a CDR H3 sequence of SEQ ID NO:12 (FIG. 1B) from an anti-human CD20 antibody and substantially the human consensus framework (FR) residues of the human heavy-chain subgroup III (V_{H}III). In a preferred embodiment, this antibody further comprises the H chain CDR H1 sequence of SEQ ID NO:10 and CDR H2 sequence of SEQ ID NO:11, and more preferably further comprises the L chain CDR L1 sequence of SEQ ID NO:4, CDR L2 sequence of SEQ ID NO:5, CDR L3 sequence of SEQ ID NO:6 and substantially the human consensus framework (FR) residues of the human light chain subgroup I (VI), wherein the V_{H} region may be joined to a human IgG chain constant region, wherein the region may be, for example, IgG1 or IgG3. See also WO 2004/056312 (Lowman et al.).

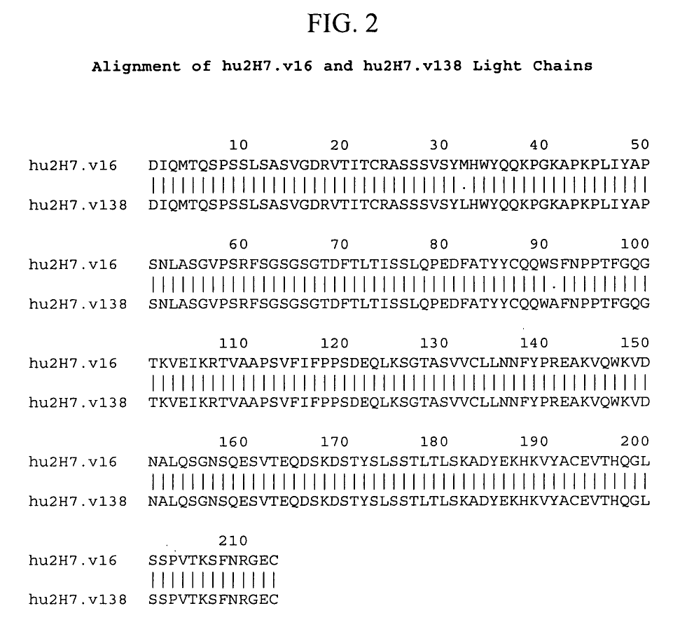

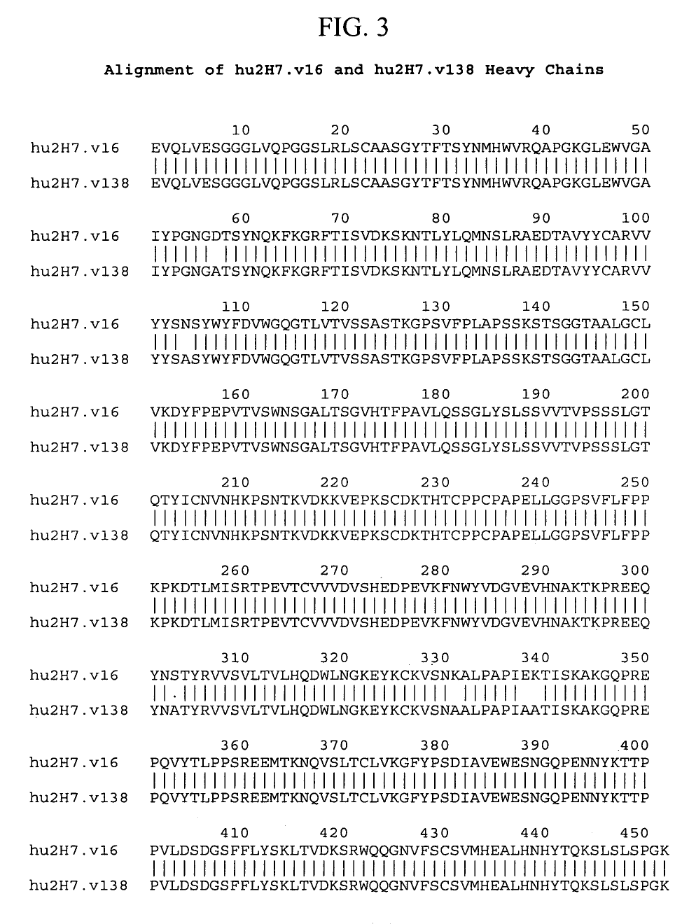

In a preferred embodiment, such antibody comprises the V_{H} sequence of SEQ ID NO:8 (v16, as shown in FIG. 1B), optionally also comprising the V_{L} sequence of SEQ ID NO:2 (v16, as shown in FIG. 1A), which may have the amino acid substitutions of D56A and N100A in the H chain and S92A in the L chain (v96). Preferably, the antibody is an intact antibody 2H7.v16 comprising the light- and heavy-chain amino acid sequences of SEQ ID NOS:13 and 14, respectively. Another preferred embodiment is where the antibody is 2H7.v31 comprising the light- and heavy-chain amino acid sequences of SEQ ID NOS:13 and 15, respectively. The antibody herein may further comprise at least one amino acid substitution in the Fc region that improves ADCC and/or CDC activity, such as one wherein the amino acid substitutions are S298A/E333A/K334A, more preferably 2H7.v31 having the heavy chain amino acid sequence of SEQ ID NO:15. Another preferred embodiment is where the antibody is 2H7.v138 comprising the light- and heavy-chain amino acid sequences of SEQ ID NOS:28 and 29, respectively, as shown in FIGS. 2 and 3, which are alignments of such sequences with the corresponding light- and heavy-chain amino acid sequences of 2H7.v16. Alternatively, such preferred intact humanized 2H7 antibody is 2H7.v477, which has the light- and heavy-chain sequences of 2H7.v138 except for the amino acid substitution of N434W. Any of these antibodies may further comprise at least one amino acid substitution in the Fc region that decreases CDC activity, for example, comprising at least the substitution K322A. See U.S. Pat. No. 6,528,624B1 (Idusogie et al.).

Some preferred humanized 2H7 variants are those having the variable light-chain domain of SEQ ID NO:2 and the variable heavy-chain domain of SEQ ID NO:8, i.e., those with or without substitutions in the Fc region, and those having a variable heavy-chain domain with alteration N100A or D56A and N100A in SEQ ID NO:8 and a variable light-chain domain with alteration M32L, or S92A, or M321, and S92A in SEQ ID NO:2, i.e., those with or without substitutions in the Fc region. If substitutions are made in the Fc region, they are preferably one of those set forth in the table below.

In a summary of some various preferred embodiments of the invention, the V region of variants based on 2F17 version 16 will have the amino acid sequences of v16 except at the positions of amino acid substitutions that are indicated in the table below. Unless otherwise indicated, the 2H7 variants will have the same L chain as that of v16.

| 2H7 | Heavy chain | Light chain | |
|---|---|---|---|
| version | (V_{H}) changes | (V_{L}) changes | Fc changes |
| 16 | | | — |
| 31 | — | — | S298A, E333A, K334A |
| 73 | N100A | M32L | |
| 75 | N100A | M32L | S298A, E333A, K334A |
| 96 | D56A, | S92A | |
| | N100A | | |
| 114 | D56A, | M32L, S92A | S298A, E333A, K334A |
| | N100A | | |
| 115 | D56A, | M32L, S92A | S298A, E333A, K334A, |
| | N100A | | E356D, M358L |
| 116 | D56A, | M32L, S92A | S298A, K334A, K322A |
| | N100A | | |
| 138 | D56A, | M32L, S92A | S298A, E333A, K334A, |
| | N100A | | K326A |
| 477 | D56A, | M32L, S92A | S298A, E333A, K334A, |
| | N100A | | K326A, N434W |
| 375 | — | — | K334L |

A particularly preferred humanized 2H7 is an intact antibody or antibody fragment comprising the variable light-chain sequence:

(SEQ ID NO:2)

DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKR;

and the variable heavy-chain sequence:

(SEQ ID NO:8)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSS.

Where the humanized 2H7 antibody is an intact antibody, preferably it comprises the light-chain amino acid sequence:

(SEQ ID NO:13)

DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC;

and the heavy-chain amino acid sequence:

(SEQ ID NO:14)

EVQLVESGGGLVQPGGSLRLSCAASGYTFTSYNNHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDCSFFLYSKLTVDKSRWQQGNVFSCSVMHEALHNHYTQKSLSLSP
GK

or the heavy-chain amino acid sequence:

(SEQ ID NO:15)

EVQLVESGGGLVQPGGSLRLSCAASGYTFTSYNMHWVRQAPGKGLEWVGA
IYPGNGDTSYNQKFKGRFTISVDKSKNTLYLQMNSLPAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNKALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

In another preferred embodiment, the intact humanized 2H7 antibody comprises the light-chain amino acid sequence:

(SEQ ID NO:28)

DTQMTQSPSSLSASVGDRVTITCPASSSVSYLHWYQQKPGKAPKPLIYAP
SNLASGVPSRFSGSGSGTDFTLTISSLQPEDFATYYCQQWAFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

and the heavy-chain amino acid sequence:

(SEQ ID NO:29)

EVQLVESGGGLVQPGGSLRLSCAASGYTFTSYNNHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

In another preferred embodiment, the humanized 2H7 antibody comprises the light-chain variable region (V_{L}) sequence of SEQ ID NO:30 and the heavy-chain variable region (V_{H}) sequence of SEQ ID NO:8, wherein the antibody further contains an amino acid substitution of D56A in VH-CDR2, and N100 in VH-CDR3 is substituted with Y or W, wherein SEQ ID NO:30 has the sequence:

(SEQ ID NO:30)

DIQMTQSPSSLSASVGDRVTITCPASSSVSYLHWYQQKPGKAPKPLIYAP
SNLASGVPSRFSGSGSGTDFTLTISSLQPEDFATYYCQQWAFNPPTFGQG
TKVEIKR.

In one embodiment of this lattermost humanized 2H7 antibody, N100 is substituted with Y. In another embodiment, N100 is substituted with W. Moreover, in a further embodiment, the antibody comprises the substitution S100aR in VH-CDR3, preferably further comprising at least one amino acid substitution in the Fc region that improves ADCC and/or CDC activity, such as one that comprises an IgG1 Fc comprising the amino acid substitutions S298A, E333A, K334A, K326A. Alternatively, the antibody comprises the substitution S100aR in VH-CDR3, preferably further comprising at least one amino acid substitution in the Fc region that improves ADCC but decreases CDC activity, such as one that comprises at least the amino acid substitution K322A, as well as one that further comprises the amino acid substitutions S298A, E333A, K334A.

In one especially preferred embodiment, the antibody comprises the 2H7.v511 light chain:

(SEQ ID NO:31)

DIQMTQSPSSLSASVGDRVTITCPASSSVSYLHWYQQKPGKAPKPLIYAP
SNLASGVPSRFSGSGSGTDFTLTISSLQPEDFATYYCQQWAFNPPTFGQG
TKVETKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

and the 2H7.v511 heavy chain:

(SEQ ID NO. 32)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

“Antibody-dependent cell-mediated cytotoxicity” and “ADCC” refer to a cell-mediated reaction in which nonspecific cytotoxic cells that express Fe receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol. 9:457-492 (1991). To assess ADCC activity of a molecule of interest, an in vitro ADCC assay, such as that described in U.S. Pat. No. 5,500,362 or U.S. Pat. No. 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo. e.g., in a animal model such as that disclosed in Clynes et al. PNAS (USA) 95:652-656 (1998).

“Human effector cells” are leukocytes that express one or more FcRs and perform effector functions. Preferably, the cells express at least FcγRIII and carry out ADCC effector function. Examples of human leukocytes that mediate ADCC include peripheral blood mononuclear cells (PBMC), natural killer (NK) cells, monocytes, cytotoxic T cells and neutrophils; with PBMCs and NK cells being preferred.

The terms “Fc receptor” or “FcR” are used to describe a receptor that binds to the Fc region of an antibody. The preferred FcR is a native-sequence human FcR. Moreover, a preferred FcR is one that binds an IgG antibody (a gamma receptor) and includes receptors of the FcγRI, FcγRII, and Fcγ RIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-492 (1991); Capel et al., Immunomethods 4:25-34 (1994); and de Haas et al., J. Lab. Clin. Med. 126:330-341 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al. J. Immunol. 24:249 (1994)).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

“Growth-inhibitory” antibodies are those that prevent or reduce proliferation of a cell expressing an antigen to which the antibody binds. For example, the antibody may prevent or reduce proliferation of B cells in vitro and/or in vivo.

Antibodies that “induce apoptosis” are those that induce programmed cell death, e.g. of a B cell, as determined by standard apoptosis assays, such as binding of annexin V, fragmentation of DNA, cell shrinkage, dilation of endoplasmic reticulum, cell fragmentation, and/or formation of membrane vesicles (called apoptotic bodies).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Imnunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains that enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc. and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256:495 (1975); Harlow et al., Antibodies. A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5):1073-1093 (2004); Fellouse, Proc. Nat. Acad. Sci. USA 101(34):12467-12472 (2004); and Lee et al. J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806; 5,569,825; 5,591,669 (all of GenPharm); U.S. Pat. No. 5,545,807; WO 1997/17852; U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody that are responsible for antigen binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

A “naked antibody” is an antibody (as herein defined) that is not conjugated to a heterologous molecule, such as a cytotoxic moiety or radiolabel.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

A “mammal” for purposes of treatment refers to any animal classified as a mammal including humans, domestic and farm animals, and zoo, sports, or pet animals, such as dogs, horses, cats, cows, etc. Preferably, the mammal is human. Such mammal is a subject, including a patient, eligible for treatment for a bone disorder who is experiencing or has experienced one or more signs, symptoms, or other indicators of a bone disorder, has been diagnosed with a bone disorder, whether, for example, newly diagnosed or previously diagnosed and now experiencing a recurrence or relapse, or is at risk for developing a bone disorder. The mammal may have been previously treated with CD20 antibody or not so treated. A mammal eligible for treatment of a bone disorder may optionally be identified as one who has been screened, as in the blood, for elevated levels of infiltrating CD20 cells.

“Treatment” of a mammal herein refers to both therapeutic treatment and prophylactic or preventative measures. Those in need of treatment include those already with a bone disorder as well as those in which the bone disorder is to be prevented. Hence, the mammal may have been diagnosed as having the bone disorder or may be predisposed or susceptible to the bone disorder. The term “treating”, “treat” or “treatment” as used herein includes preventative (e.g., prophylactic), palliative and curative treatment. The methods of this invention result in bone formation resulting in decreased fracture rates. This invention makes a significant contribution to the art by providing methods that increase bone formation resulting in prevention, retardation, and/or regression of osteoporosis and related bone disorders.

A “symptom” of a bone disorder is any morbid phenomenon or departure from the normal in structure, function, or sensation, experienced by the mammal and indicative of disease, such as those noted above.

The phrase “agent that treats osteoclast-associated disorders” refers to any molecule that can be used to inhibit, prevent, treat, or lessen the symptoms of a bone disorder as defined herein. Examples include growth factors and other therapeutic agents that have a positive effect on the growth of bone or connective tissue, such as growth factors including insulin-like growth factor 1 (IGF-1), PDGF, an inhibitor of alpha-transforming growth factor (TGF-α), beta-transforming growth factor (TGF-β), epidermal growth factor (EGF), bone morphogenetic proteins, leukemia inhibitory factor, fibroblast growth factors, cytokines such as interleukin-4 (IL-4) and mutants of IL-4 as described in US 2004/0126364, a zvegf3 protein as described in US 2004/0043031 and U.S. Pat. No. 6,663,870, and zvegf4 proteins, as well as other therapeutic agents, including vitamin D, bisphosphonates, calcitonin, estrogens, parathyroid hormone, osteogenin, NaF, osteoprotegerin, statins, a TRANCE/RANK inhibitor effective to inhibit osteoclast bone erosion activity as described by U.S. Pat. Nos. 6,682,739B1 and 6,673,771B1, beta glucans for treatment of osteoporosis and other diseases of bone resorption as described in US20040058889, one or more compounds known in the art to be beneficial to bone formation, such as calcium, fluoride, magnesium, boron, or a combination thereof; thienyl-substituted acylguanidines as inhibitors of bone resorption and vitronectin receptor antagonists as described in U.S. Pat. No. 6,660,728; acylquanidine derivatives as described in U.S. Pat. No. 6,602,878; a matrix selected from the group consisting of glycolic acid, lactic acid, collagen, demineralized bone, or a combination thereof. A first biologically active molecule comprising a fibronectin is attached to a portion of the matrix, to facilitate osteoblast activity and for promoting an increase in bone formation. A second biologically active molecule comprising a vitronectin, selected for its ability to attract osteoclasts and produce an inhibiting effect on osteoclast activity to thereby promote a decrease in bone resorption, is also attached to a portion of the matrix as described in US 2003/0219429; plasminogen activator inhibitors, of which there are two classes. These two classes are SERPINs (serine protease inhibitors) and nexin I. There are two types of inhibitors within the class of SERPINs, namely, PAI-1 and PAI-2. PAI-1 exists in both latent and active forms; proteoglycans; fibronectin and fibronectin fragments; vitronectin and vitronectin fragments; collagen and collagen fragments; heparin and heparin fragments; von Willebrand factor; bone sialoprotein; osteopontin; osteonectin; osteocalcin; selectin and selectin fragments; proteins and peptides that facilitate cell adhesion (including cyclic versions): RGD-type (Arg-Gly-Asp) and GPR (Gly-Pro-Arg) etc.; GHK-type (Gly-His-Lys) etc.; laminin or laminin-fragment etc.; EIL-type (Glu-Ile-Leu) etc.; LDV-type (Leu-Asp), LDV-NH2 (Leu-Asp-Val-NH2) etc.; synthetic peptides containing the RGD or GHK sequence of amino acids; osteonectin and SPARC (Secreted Protein Acidic and Rich in Cysteine); osteopontin; collagens, Type I and Type II; von Willebrand Factor (a glycoprotein that facilitates adhesion of cells to structures. It has an ability to link to cells and thus the potential of being a ligand for cell surface receptors of osteoblasts); bone sialoprotein; thrombospondin; osteocalcin; cytomodulin; bone morphogenetic proteins (BMPs); tenascins; fibrinolysis inhibiting factor; growth factors, e.g., platelet-derived growth factors (PDGF), insulin-like growth factors (IGFs), etc.; antibodies to cell surface components e.g., β-1; integrin antibody; plasminogen activator inhibitors (PAIs); protease inhibitors; and metalloprotease inhibitors. (In the above description, the conventional notation is followed whereby: R=Arg, G=Gly, D=Asp, S=Ser, C=Cys, V=Val, E=Glu, A=Ala, P=Pro, K=Lys, T=Thr, Y=Tyr, Q=Gln, H=His, L=Leu, I=Ile and F=Phe.) Preferred are an osteoprotegerin, an interleukin, a MMP inhibitor, a beta glucan, an integrin antagonist, calcitonin, a proton pump inhibitor, a protease inhibitor, a bisphosphonate, insulin-like growth factor-1, platelet-derived growth factor, epidermal growth factor, an inhibitor of transforming growth factor-alpha, transforming growth factor-beta, a bone morphogenetic protein, parathyroid hormone, osteoprotegerin, a fibroblast growth factor, Vitamin D, calcium, fluoride, magnesium, or boron, vitronectin, plasminogen-activator inhibitor, or protease inhibitor such as a metalloprotease inhibitor. More preferred is wherein the agent is a cytokine or bisphosphonate.

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROL® methylprednisolone sodium succinate, and dexamethasone; dihydrofolate reductase inhibitors such as methotrexate (oral or subcutaneous); anti-malarial agents such as chloroquine and hydroxychloroquine; sulfasalazine; leflunomide; cytokine or cytokine receptor antibodies including anti-interferon-alpha, -beta, or -gamma antibodies, anti-tumor necrosis factor (TNF)-alpha antibodies (infliximab (REMICADE®) or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; transforming growth factor-beta (TGF-β); streptodomase; RNA or DNA from the host; FK506; RS-61443; chlorambucil; deoxyspergualin; rapamycin; T-cell receptor (Cohen et al., U.S. Pat. No. 5,114,721); T-cell receptor fragments (Offner et al., Science, 251: 430-432 (1991); WO 90/11294; Ianeway, Nature, 341: 482 (1989); and WO 91/01133); BAFF antagonists such as BAFF antibodies and BR3 antibodies and zTNF4 antagonists (for review, see Mackay and Mackay, Trends Immunol., 23:113-5 (2002) and see also definition below); biologic agents that interfere with F cell helper signals, such as anti-CD40 receptor or anti-CD40 ligand (CD154), including blocking antibodies to CD40-CD40 ligand (e.g., Durie et al., Science, 261: 1328-30 (1993); Mohan et al., J. Immunol., 154: 1470-80 (1995)) and CTLA4-Ig (Finck et al., Science, 265: 1225-7 (1994)); and T-cell receptor antibodies (EP 340,109) such as T10B9. Some preferred immunosuppressive agents herein include cyclophosphamide, chlorambucil, azathioprine, leflunomide, MMF, or methotrexate.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small-molecule toxins or enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII (see, e.g., Angew, Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN®) vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); and pharmaceutically acceptable salts, acids or derivatives of any of the above.

The term “cytokine” is a generic term for proteins released by one cell population that act on another cell as intercellular mediators. Examples of such cytokines are lymphokines; monokines; interleukins (ILs) such as IL-1, IL-1α, IL-2, IL-3, IL-4, IL-5, IL-6, IL-7, IL-8; IL-9, IL-11, IL-12, IL-15, including PROLEUKIN® rIL-2 and human IL-4 and mutants of human IL-4, such as, for example, a mutant containing a mutation in the region of IL-4 which is involved in binding to IL-2R gamma, e.g., Arg 21 is changed to a Glu residue; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof.

The term “hormone” refers to polypeptide hormones, which are generally secreted by glandular organs with ducts. Included among the hormones are, for example, growth hormone such as human growth hormone, N-methionyl human growth hormone, and bovine growth hormone; parathyroid hormone; thyroxine; insulin; proinsulin; relaxin; estradiol; hormone-replacement therapy; androgens such as calusterone, dromostanolone propionate, epitiostanol, mepitiostane, or testolactone; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, gonadotropin-releasing hormone; inhibin; activin; mullerian-inhibiting substance; and thrombopoietin. As used herein, the term hormone includes proteins from natural sources or from recombinant cell culture and biologically active equivalents of the native-sequence hormone, including synthetically produced small-molecule entities and pharmaceutically acceptable derivatives and salts thereof.

The term “growth factor” refers to proteins that promote growth, and include, for example, hepatic growth factor; fibroblast growth factor; vascular endothelial growth factor; nerve growth factors such as NGF-β; platelet-derived growth factor; transforming growth factors (TGFs) such as TGF-α and TGF-β; insulin-like growth factor-I and -II; erythropoietin (EPO); osteoinductive factors; interferons such as interferon-α, -β, and -γ; and colony stimulating factors (CSFs) such as macrophage-CSF (M-CSF); granulocyte-macrophage-CSF (GM-CSF); and granulocyte-CSF (G-CSF). As used herein, the term growth factor includes proteins from natural sources or from recombinant cell culture and biologically active equivalents of the native-sequence growth factor, including synthetically produced small-molecule entities and pharmaceutically acceptable derivatives and salts thereof.

The term “integrin” refers to a receptor protein that allows cells both to bind to and to respond to the extracellular matrix and is involved in a variety of cellular functions such as wound healing, cell differentiation, homing of tumor cells and apoptosis. They are part of a large family of cell adhesion receptors that are involved in cell-extracellular matrix and cell-cell interactions. Functional integrins consist of two transmembrane glycoprotein subunits, called alpha and beta, that are non-covalently bound. The alpha subunits all share some homology to each other, as do the beta subunits. The receptors always contain one alpha chain and one beta chain. Examples include Alpha6beta1, Alpha3beta1, Alpha7beta1, LFA-1 etc. As used herein, the term “integrin” includes proteins from natural sources or from recombinant cell culture and biologically active equivalents of the native-sequence integrin, including synthetically produced small-molecule entities and pharmaceutically acceptable derivatives and salts thereof.

For the purposes herein. “tumor necrosis factor alpha (TNF-α)” refers to a human TNF-α molecule comprising the amino acid sequence as described in Pennica et al., Nature, 312:721 (1984) or Aggarwal et al., JBC, 260:2345 (1985). A “TNF-α inhibitor” herein is an agent that inhibits, to some extent, a biological function of TNF-α, generally through binding to TNF-α and neutralizing its activity. Examples of TNF inhibitors specifically contemplated herein are etanercept (ENBREL®), infliximab (REMICADE®), and adalimumab (HUMIRA™).

Examples of “disease-modifying anti-rheumatic drugs” or “DMARDs” include hydroxycloroquine, sulfasalazine, methotrexate, leflunomide, etanercept, infliximab (plus oral and subcutaneous methotrexate), azathioprine, D-penicillamine, gold salts (oral), gold salts (intramuscular), minocycline, cyclosporine including cyclosporine A and topical cyclosporine, staphylococcal protein A (Goodyear and Silverman, J. Exp. Med., 197, (9), p1125-39 (2003)), including salts and derivatives thereof, etc.

Examples of “non-steroidal anti-inflammatory drugs” or “NSAIDs” include aspirin, acetylsalicylic acid, ibuprofen, naproxen, indomethacin, sulindac, tolmetin, COX-2 inhibitors such as celecoxib (CELEBREX®; 4-(5-(4-methylphenyl)-3-(trifluoromethyl)-1H-pyrazol-1-yl)benzenesulfonamide and valdecoxib (BEXTRA®), and meloxicam (MOBIC®), including salts and derivatives thereof, etc. Preferably, they are aspirin, naproxen, ibuprofen, indomethacin, or tolmetin.

Examples of “integrin antagonists or antibodies” herein include an LFA-1 antibody, such as efalizumab (RAPTIVA®) commercially available from Genentech, or an alpha 4 integrin antibody such as natalizumab (TYSABRI®) available from Biogen Idec, or diazacyclic phenylalanine derivatives (WO 2003/89410), phenylalanine derivatives (WO 2003/70709, WO 2002/28830, WO 2002/16329 and WO 2003/53926), phenylpropionic acid derivatives (WO 2003/10135), enamine derivatives (WO 2001/79173), propanoic acid derivatives (WO 2000/37444), alkanoic acid derivatives (WO 2000/32575), substituted phenyl derivatives (U.S. Pat. Nos. 6,677,339 and 6,348,463), aromatic amine derivatives (U.S. Pat. No. 6,369,229), ADAM disintegrin domain polypeptides (US2002/0042368), antibodies to alphavbeta3 integrin (EP 633945), aza-bridged bicyclic amino acid derivatives (WO 2002/02556), etc.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone, such as SOLU-MEDROL® methylprednisolone sodium succinate), dexamethasone or dexamethasone triamcinolone, hydrocortisone, and betamethasone. The preferred corticosteroids herein are prednisone, methylprednisolone, hydrocortisone, or dexamethasone.

The terms “BAFF,” “BAFF polypeptide,” “TALL-1” or “TALL-1 polypeptide,” and “BLyS” when used herein encompass “native-sequence BAFF polypeptides” and “BAFF variants”. “BAFF” is a designation given to those polypeptides that have any one of the amino acid sequences shown below:

Human BAFF sequence (SEQ ID NO:16):

| | |
|---|---|
| 1 | MDDSTEREQSRLTSCLKKREEMKLKECVSILPRKESPSVRSSKDGKL |
| | LAATLLLALLSCC |
| 61 | LTVVSFYQVAALQGDLASLPAELQGHHAEKLPAGAGAPKAGLEEAPA |
| | VTAGLKIFEPPAP |
| 121 | GEGNSSQNSRNKRAVQGPEETVTQDCLQLIADSETPTIQKGSYTFVP |
| | WLLSFKRGSALEE |
| 181 | KENKILVKETGYFFIYGQVLYTDKTYAMGHLIQRKKVHVFGDELSLV |
| | TLFRCIQNMPETL |
| 241 | PNNSCYSAGIAKLEEGDELQLAIPRENAQISLDGDVTFFGALKLL |

Mouse BAFF sequence (SEQ ID NO:17):

| | |
|---|---|
| 1 | MDESAKTLPPPCLCFCSEKGEDMKVGYDPITPQKEEGAWFGTCRDGR |
| | LLAATLLLALLSS |
| 61 | SFTANSLYQLAALQADLMNLRMELQSYRGSATPAAAGAPELTAGVKL |
| | LTPAAPRPHNSSR |
| 121 | GHRNRRAFQGPEETEQDVDLSAPPAPCLPGCRHSQHDDNGMNLRNII |
| | QDCLQLIADSDTP |
| 181 | TIRKGTYTFVPWLLSFKRGNALEEKENKIVVRQTGYFFIYSQVLYTD |
| | PIFAMGHVIQRKK |
| 241 | VHVFGDELSLVTLFRCIQNMPKTLPNNSCYSAGIARLEEGDEIQLAI |
| | PRENAQISRNGDD |
| 301 | TFFGALKLL |

and homologs and fragments and variants thereof, which have the biological activity of the native BAFF. A biological activity of BAFF can be selected from the group consisting of promoting B cell survival, promoting B cell maturation and binding to BR3. Variants of BAFF will preferably have at least 80% or any successive integer up to 100% including, more preferably, at least 90%, and even more preferably, at least 95% amino acid sequence identity with a native sequence of a BAFF polypeptide.

A “native-sequence” BAFF polypeptide comprises a polypeptide having the same amino acid sequence as the corresponding BAFF polypeptide derived from nature. For example, BAFF exists in a soluble form following cleavage from the cell surface by furin-type proteases. Such native-sequence BAFF polypeptides can be isolated from nature or can be produced by recombinant and/or synthetic means.

The term “native-sequence BAFF polypeptide” or “native BAFF” specifically encompasses naturally occurring truncated or secreted forms (e.g., an extracellular domain sequence), naturally occurring variant forms (e.g., alternatively spliced forms), and naturally occurring allelic variants of the polypeptide. The term “BAFF” includes those polypeptides described in Shu et al., J. Leukocyle Biol., 65:680 (1999); GenBank Accession No. AF136293; WO 1998/18921 published May 7, 1998; EP 869,180 published Oct. 7, 1998; WO 1998/27114 published Jun. 25, 1998; WO 1999/12964 published Mar. 18, 1999; WO 1999/33980 published Jul. 8, 1999; Moore et al., Science, 285:260-263 (1999); Schneider et al., J. Exp. Med., 189:1747-1756 (1999) and Mukhopadhyay et al., J. Biol. Chem., 274:15978-15981 (1999).

The term “BAFF antagonist” as used herein is used in the broadest sense, and includes any molecule that (1) binds a native-sequence BAFF polypeptide or binds a native-sequence of BR3 to partially or fully block BR3 interaction with BAFF polypeptide, and (2) partially or fully blocks, inhibits, or neutralizes native-sequence BAFF activity. In one preferred embodiment the BAFF receptor to be blocked is the BR3 receptor. Native BAFF activity promotes, among other things, B-cell survival and/or B-cell maturation. In one embodiment, the inhibition, blockage or neutralization of BAFF activity results in a reduction in the number of B cells. A BAFF antagonist according to this invention will partially or fully block, inhibit, or neutralize one or more biological activities of a BAFF polypeptide, in vitro and/or in vivo. In one embodiment, a biologically active BAFF potentiates any one or a combination of the following events in vitro and/or in vivo: an increased survival of B cells, an increased level of IgG and/or IgM, an increased numbers of plasma cells, and processing of NF-κb2/100 to p52 NF-κb in splenic B cells (e.g., Batten et al., J. Exp. Med. 192:1453-1465 (2000); Moore et al., Science 285:260-263 (1999); Kayagaki et al. Immunity 17:515-524 (2002)).

As mentioned above, a BAFF antagonist can function in a direct or indirect manner to partially or fully block, inhibit or neutralize BAFF signaling, in vitro or in vivo. For instance, the BAFF antagonist can directly bind BAFF. For example, BAFF antibodies that bind within a region of human BAFF comprising residues 162-275 and/or a neighboring residue of a residue selected from the group consisting of 162, 163, 206, 211, 231, 233, 264 and 265 of human BAFF such that the antibody sterically hinders BAFF binding to BR3 are contemplated, where such residue numbers refer to SEQ ID NO:16. In another example, a direct binder is a polypeptide comprising any portion of a BAFF receptor that binds BAFF such as an extracellular domain of a BAFF receptor, or fragments and variants thereof that bind native BAFF. In another example, BAFF antagonists include the polypeptides having a sequence of a polypeptide comprising the sequence of Formula I:
X₁-C-X₃-D-X₅-L-X₇-X₈-X₉-X₁₀-X₁₁-X₁₂-C-X₁₄-X₁₅-X₁₆-X₁₇
(Formula I) (SEQ ID NO:18)
wherein X1, X3, X5, X7, X8, X9, X10, X11, X12, X14, X15 and X17 are any amino acid except cysteine; and
wherein X16 is an amino acid selected from the group consisting of L, F, I and V; and
wherein the polypeptide does not comprise a cysteine within seven amino acid residues N-terminal to the most N-terminal cysteine C and C-terminal to the most C-terminal cysteine C of Formula I.

In one embodiment, a polypeptide comprising the sequence of Formula I has the two Cs joined by disulfide bonding; X₅LX₇X₈ forming the conformation of a type I beta turn structure with the center of the turn between L and X₇; and has a positive value for the dihedral angle phi of X₈. In one embodiment, X₁₀ is selected from the group consisting of W, F, V, L, I, Y, M and a non-polar amino acid. In another embodiment, X₁₀ is W. In another embodiment, X₃ is an amino acid selected from the group consisting of M, V, L, I, Y, F, W and a non-polar amino acid. In another embodiment, X₅ is selected from the group consisting of V, L, P, S, I, A and R. In another embodiment, X₇ is selected from the group consisting of V, T, I and L. In another embodiment, X₈ is selected from the group consisting of R, K, G, N, H and a D-amino acid. In another embodiment, X₉ is selected from the group consisting of H, K, A, R and Q. In another embodiment, X₁₁ is I or V. In another embodiment, X₁₂ is selected from the group consisting of P, A, D. E and S. In another embodiment, X₁₆ is L. In one specific embodiment, the sequence of Formula I is a sequence selected from the group consisting of ECFDLLVRAWVPCSVLK (SEQ ID NO:19), ECFDLLVRHWVPCGLLR (SEQ ID NO:20), ECFDLLVRRWVPCEMLG (SEQ ID NO:21), ECFDLLVRSWVPCHMLR (SEQ ID NO:22), ECFDLLVRHWVACGLLR (SEQ ID NO:23), and QCFDRLNAWVPCSVLK (SEQ ID NO:24). In a preferred embodiment, the BAFF antagonist comprises any one of the amino acid sequences selected from the group consisting of SEQ ID NO:19, 20, 21, 22, and 23.

In still another example, BAFF antagonists include the polypeptides having a sequence of a polypeptide comprising the sequence of Formula II:
X₁-C-X₃-D-X₅-L-V-X₈-X₉-W-V-P-C-X₁₄-X₁₅-L-X₁₇
(Formula II) (SEQ ID NO:25)
wherein X1, X3, X5, X8, X9, X14, X15 and X17 are any amino acid, except cysteine; and wherein the polypeptide does not comprise a cysteine within seven amino acid residues N-terminal to the most N-terminal cysteine C and C-terminal to the most C-terminal cysteine C of Formula II.

In one embodiment, a polypeptide comprising the sequence of Formula II has a disulfide bond between the two Cs and has the conformation of X₅LX₇X8 forming a type I beta turn structure with the center of the turn between L and X₇; and has a positive value for the dihedral angle phi of X₈. In another embodiment of Formula II, X₃ is an amino acid selected from the group consisting of M, A, V, L, I, Y, F, W and a non-polar amino acid. In another embodiment of Formula II, X₅ is selected from the group consisting of V, L, P, S, I, A and R. In another embodiment of Formula II, X₈ is selected from the group consisting of R, K, G, N, H and D-amino acid. In another embodiment of Formula II, X₉ is selected from the group consisting of H, K, A, R and Q.

In a further embodiment, the BAFF receptor from which the extracellular domain or BAFF-binding fragment or BAFF-binding variant thereof is derived is TACI, BR3 or BCMA. Alternatively, the BAFF antagonist can bind an extracellular domain of a native-sequence BR3 at its BAFF binding region to partially or fully block, inhibit or neutralize BAFF binding to BR3 in vitro, in situ, or in vivo. For example, such indirect antagonist is an anti-BR3 antibody that binds in a region of BR3 comprising residues 23-38 of human BR3 as defined below (SEQ ID NO:26) or a neighboring region of those residues such that binding of human BR3 to BAFF is sterically hindered.

In some embodiments, a BAFF antagonist according to this invention includes BAFF antibodies and immunoadhesins comprising an extracellular domain of a BAFF receptor, or fragments and variants thereof that bind native BAFF. In a further embodiment, the BAFF receptor from which the extracellular domain or BAFF-binding fragment or BAFF-binding variant thereof is derived is TACI, BR3 or BCMA. In a still another embodiment, the immunoadhesin comprises an amino acid sequence of that of Formula I or Formula II as set forth above, including an amino acid sequence selected from any one of the group consisting of SEQ ID NOS: 19, 20, 21, 22, 23, and 24.

According to one embodiment, the BAFF antagonist binds to a BAFF polypeptide or a BR3 polypeptide with a binding affinity of 100 nM or less. According to another embodiment, the BAFF antagonist binds to a BAFF polypeptide or a BR3 polypeptide with a binding affinity of 10 nM or less. According to yet another embodiment, the BAFF antagonist binds to a BAFF polypeptide or a BR3 polypeptide with a binding affinity of 1 nM or less.

The terms “BR3”, “BR3 polypeptide” or “BR3 receptor” when used herein encompass “native-sequence BR3 polypeptides” and “BR3 variants” (which are further defined herein). “BR3” is a designation given to those polypeptides comprising the following amino acid sequence and homologs thereof, and variants or fragments thereof that bind native BAFF:

Human BR3 sequence (SEQ ID NO:26):

| | |
|---|---|
| 1 | MRRGPRSLRGRDAPAPTPCVPAECFDLLVRHCVACGLLRTPRPKPAG |
| | ASSPAPRTALQPQ |
| 61 | ESVGAGAGEAALPLPGLLFGAPALLGLALVLALVLVGLVSWRRRQRR |
| | LRGASSAEAPDGD |
| 121 | KDAPEPLDKVIILSPGISDATAPAWPPPGEDPGTTPPGHSVPVPATE |
| | LGSTELVTTKTAG |
| 181 | PEQQ |

The BR3 polypeptides of the invention can be isolated from a variety of sources, such as from human tissue types or from another source, or prepared by recombinant and/or synthetic methods. The term BR3 includes the BR3 polypeptides described in WO 2002/24909 and WO 2003/14294.

A “native-sequence” BR3 polypeptide or “native BR3” comprises a polypeptide having the same amino acid sequence as the corresponding BR3 polypeptide derived from nature. Such native-sequence BR3 polypeptides can be isolated from nature or can be produced by recombinant and/or synthetic means. The term “native-sequence BR3 polypeptide” specifically encompasses naturally occurring truncated, soluble or secreted forms (e.g., an extracellular domain sequence), naturally occurring variant forms (e.g., alternatively spliced forms) and naturally occurring allelic variants of the polypeptide. The BR3 polypeptides of the invention include the BR3 polypeptide comprising or consisting of the contiguous sequence of amino acid residues 1 to 184 of a human BR3 (SEQ ID NO:26).

A BR3 “extracellular domain” or “ECD” refers to a form of the BR3 polypeptide that is essentially free of the transmembrane and cytoplasmic domains. ECD forms of BR3 include a polypeptide comprising any one of the amino acid sequences selected from the group consisting of amino acids 1-77, 2-62, 2-71, 1-61, 7-71, 23-38 and 2-63 of human BR3. The invention contemplates BAFF antagonists that are polypeptides comprising any one of the above-mentioned ECD forms of human BR3 and variants and fragments thereof that bind a native BAFF.

Mini-BR3 is a 26-residue core region of the BAFF-binding domain of BR3, i.e., the amino acid sequence: TPCVPAECFD LLVRHCVACG LLRTPR (SEQ ID NO:27)

“BR3 variant” means a BR3 polypeptide having at least about 80% amino acid sequence identity with the amino acid sequence of a native-sequence, full-length BR3 or BR3 ECD and binds a native-sequence BAFF polypeptide. Optionally, the BR3 variant includes a single cysteine-rich domain. Such BR3 variant polypeptides include, for instance, BR3 polypeptides wherein one or more amino acid residues are added, or deleted, at the N- and/or C-terminus, as well as within one or more internal domains, of the full-length amino acid sequence. Fragments of the BR3 ECD that bind a native sequence BAFF polypeptide are also contemplated. According to one embodiment, a BR3 variant polypeptide will have at least about 80% amino acid sequence identity, at least about 81% amino acid sequence identity, at least about 82% amino acid sequence identity, at least about 83% amino acid sequence identity, at least about 84% amino acid sequence identity, at least about 85% amino acid sequence identity, at least about 86% amino acid sequence identity, at least about 87% amino acid sequence identity, at least about 88% amino acid sequence identity, at least about 89% amino acid sequence identity, at least about 90% amino acid sequence identity, at least about 91% amino acid sequence identity, at least about 92% amino acid sequence identity, at least about 93% amino acid sequence identity, at least about 94% amino acid sequence identity, at least about 95% amino acid sequence identity, at least about 96% amino acid sequence identity, at least about 97% amino acid sequence identity, at least about 98% amino acid sequence identity or at least about 99% amino acid sequence identity with a human BR3 polypeptide or a specified fragment thereof (e.g., ECD). BR3 variant polypeptides do not encompass the native BR3 polypeptide sequence. According to another embodiment, BR3 variant polypeptides are at least about 10 amino acids in length, at least about 20 amino acids in length, at least about 30 amino acids in length, at least about 40 amino acids in length, at least about 50 amino acids in length, at least about 60 amino acids in length, or at least about 70 amino acids in length.

In one preferred embodiment, the BAFF antagonists herein are immunoadhesins comprising a portion of BR3, TACI or BCMA that binds BAFF, or variants thereof that bind BAFF. In other embodiments, the BAFF antagonist is a BAFF antibody. A “BAFF antibody” is an antibody that binds BAFF, and preferably binds BAFF within a region of human BAFF comprising residues 162-275 of the human BAFF sequence disclosed herein under the “BAFF” definition (SEQ ID NO:16). In another embodiment, the BAFF antagonist is BR3 antibody. A “BR3 antibody” is an antibody that binds BR3, and is preferably one that binds BR3 within a region of human BR3 comprising residues 23-38 of the human BR3 sequence disclosed herein under the “BR3” definition (SEQ ID NO:26). In general, the amino acid positions of human BAFF and human BR3 referred to herein are according to the sequence numbering under human BAFF and human BR3, SEQ ID NOS: 16 and 26, respectively, disclosed herein under the “BAFF” and “BR3” definitions.

Other examples of BAFF-binding polypeptides or BAFF antibodies can be found in, e.g., WO 2002/092620, WO 2003/014294, Gordon et al., Biochemistry 42(20):5977-5983 (2003), Kelley et al., J. Biol. Chem., 279(16):16727-16735 (2004), WO 1998/18921, WO 2001/12812, WO 2000/68378 and WO 2000/40716.

A “liposome” is a small vesicle composed of various types of lipids, phospholipids and/or surfactant that is useful for delivery of a drug (such as the antagonists disclosed herein) to a mammal. The components of the liposome are commonly arranged in a bilayer formation, similar to the lipid arrangement of biological membranes.

As used herein, the term “bone disorder” refers to a disease characterized by bone loss, i.e., a disease, condition, disorder or syndrome that has as a symptom or pathology a decrease in bone mass or density. Examples of diseases characterized by bone loss include, but are not limited to, osteolysis, including osseous metastasis, aseptic prosthetic loosening, periodontitis, osteoporosis, Paget's disease, metastatic bone disease, and rheumatoid arthritis. Such bone disorders include those associated with autoimmune diseases such as lupus and rheumatoid arthritis. It has been found that women with SLE had significantly lower bone mineral density T-scores than women without elevated SLE disease damage regardless of prior corticosteroid use status. Such bone disorders also include conditions associated with low bone mass, including a condition where the level of bone mass is below the age specific normal as defined in standards by the World Health Organization “Assessment of Fracture Risk and its Application to Screening for Postmenopausal Osteoporosis (1994). Report of a World Health Organization Study Group. World Health Organization Technical Series 843”. Included in condition(s) associated with low bone mass are primary and secondary osteoporosis. Also included are periodontal disease, alveolar bone loss, post-osteotomy and childhood idiopathic bone loss, as well as long-term complications of osteoporosis such as curvature of the spine, loss of height, and prosthetic surgery. Such bone disorders may affect those who present with low bone mass, such as vertebrates, e.g., mammals, known to have a significantly higher than average chance of developing such diseases as are described above including osteoporosis (e.g., post-menopausal women, men over the age of 50). The disorder can be treated with bone-mass-augmenting or -enhancing methods, including bone restoration, increasing the bone fracture healing rate, replacing bone graft surgery entirely, enhancing the rate of successful bone grafts, bone healing following facial reconstruction or maxillary reconstruction or mandibular reconstruction, prosthetic ingrowth, vertebral synostosis or long bone extension. Those skilled in the art will recognize that the term bone mass actually refers to bone mass per unit area, which is sometimes (although not strictly correctly) referred to as bone mineral density.

Examples of bone disorders herein include osteoporosis, such as primary or secondary osteoporosis, and including glucocorticoid-induced osteoporosis, a focal bone erosion or disease such as that from rheumatoid arthritis and including marginal joint erosions and subchondral bone erosions (bone marrow), Paget's disease, a bone defect, abnormally increased bone turnover, periodontal disease, tooth loss, periprosthetic osteolysis, osteogenesis imperfecta, metastatic bone disease, hypercalcemia of malignancy, childhood idiopathic bone loss, alveolar bone loss, bone fracture, osteopenia such as juxta-articular osteopenia, bone disease in multiple myeloma and related conditions such as Waldenstroms' macroglobulinemia and/or monoclonal gammopathy. Preferred bone disorders herein are bone disease in multiple mycloma, macroglulinemia and monoclonal gammopathy and osteoporosis, more preferably secondary osteoporosis, and more preferably still bone loss during inflammation. Bone disorders that are not associated with a malignancy are also within the scope of the invention.

“Secondary osteoporosis” includes bone loss during inflammation, glucocorticoid-induced osteoporosis, hyperthyroidism-induced osteoporosis, immobilization-induced osteoporosis, heparin-induced osteoporosis and immunosuppressive-induced osteoporosis in a vertebrate, e.g., a mammal (including a human being). As used herein, the term “bone resorption” refers to the undesired loss of bone caused at least in part by osteoclast activity.

“Osteolysis” refers to catastrophic bone loss, or a debilitating pathological consequence of a spectrum of disease states including rheumatoid arthritis, osseous metastasis, aseptic prosthetic loosening and periodontitis. Rheumatoid arthritis (RA) is a chronic inflammatory disease which often results in long-term disability and increased mortality.

“Osteoprogenitor” refers to a differentiated bone precursor cell derived from a bone stromal cell.

“Odontoprogenitor” refers to a differentiated bone precursor cell derived from periodontal ligament.

As used herein, the term “inhibit” means to decrease the amount, quality, or effect of a particular activity and is used interchangeably with the terms “reduce”, “minimize”, and “lessen” and refers to, for example, the reduction of osteoclast bone erosion activity caused by the administration of a therapeutically effective amount of the compounds of the present invention to a mammal. Treating a bone disorder as defined herein includes inhibiting bone resorption, osteoclastogenesis or osteoclast function and treating a disease characterized by bone loss.

As used herein, the term “effective amount” is meant to refer to an amount of the antibody or antagonist that is effective for treating the bone disorder. Thus, in one aspect, an “effective amount” of a composition for use in treating a condition associated with bone loss or in a condition wherein promotion of bone growth is beneficial is an amount sufficient to inhibit bone loss and/or increase bone formation or to inhibit osteoclast activity. In a more specific or alternative aspect, the compound in an effective amount produces a medicinal effect observed as reduction in the rate of bone loss in an individual when a therapeutically effective amount of a compound is administered to an individual who is susceptible to or suffering from a disease characterized by bone loss. Effective amounts are typically determined by the effect they have compared to the effect observed when a composition that includes no active ingredient (i.e. a control) is administered to a similarly situated individual. Additionally, an “effective amount” of a composition is that amount that produces a statistically significant effect, such as a statistically significant increase in the rate of fracture repair, reversal of bone loss in osteoporosis, increase in the rate of healing of a joint injury, increase in the reversal of cartilage defects, increase or acceleration of bone growth into prosthetic devices, improved repair of dental defects, and the like.

A “package insert” is used to refer to instructions customarily included in commercial packages of therapeutic products that contain information about the indications, usage, dosage, administration, contraindications, other therapeutic products to be combined with the packaged product, and/or warnings concerning the use of such therapeutic products, etc.

A “medicament” is an active drug to treat the bone disorder or its symptoms or side effects.

II. Therapy

In one aspect, the present invention provides a method of treating bone disorders in a mammal comprising administering an antagonist, preferably an antibody, that binds to a B-cell surface marker (more preferably a CD20 antibody) to the mammal.

The exact dose will be determined by the clinician according to accepted standards, taking into account the nature and severity of the condition to be treated, the type of antagonist, the mammal/patient traits, etc. Determination of dose is within the level of ordinary skill in the art. Depending upon the route and method of administration, the protein may be administered in a single dose, as a prolonged infusion, or intermittently over an extended period. Intravenous administration will be by bolus injection or infusion over a typical period of one to several hours. Sustained release formulations can be employed. In general, a therapeutically effective amount of CD20 antagonist is an amount sufficient to produce a clinically significant change in the treated condition, such as a clinically significant reduction in time required for fracture repair, a significant reduction in the volume of a void or other defect, a significant increase in bone density, a significant reduction in morbidity, or a significantly increased histological score.

In a preferred embodiment, the dose is about 400 mg to 1.3 grams at a frequency of about one to four doses within a period of about one month, more preferably about 500 mg to 1.2 grams, and still more preferably about 750 mg to 1.1 grams. In another preferred embodiment, the antibody is administered in about two to four doses, more preferably in about two to three doses, and most preferably in about two doses. In a still preferred embodiment, the antibody is administered within a period of about 2 to 3 weeks, more preferably about two weeks.

The particular number of doses (whether one, two or three or more) employed is dependent, for example, on the type of bone disorder treated, the type of antibody employed, whether, what type, and how much and how many of a second medicament is employed as noted below, and the method and frequency of administration. Where more than one dose is administered, the later dose (for example, second or third dose) is preferably administered from about 1 to 20 days, more preferably from about 6 to 16 days, and most preferably from about 14 to 16 days from the time the previous dose was administered. The separate doses are preferably administered within a total period of between about 1 day and 4 weeks, more preferably between about 1 and 20 days (e.g., within a period of 6-18 days). In one such aspect, the separate doses are administered about weekly, with the second dose being administered about one week from the first dose and any third or subsequent dose being administered about one week from the second dose. Each such separate dose of the antibody is preferably about 0.5 to 1.5 grams, more preferably about 0.75 to 1.3 grams.

For local application, such as for the regeneration of bone in a fracture or other bony defect, the protein may be applied in the range of about 0.1-100 μg/cm² of wound area.

As noted above, however, these suggested amounts of antagonist are subject to a great deal of therapeutic discretion. The key factor in selecting an appropriate dose and scheduling is the result obtained, as indicated above. For example, relatively higher doses may be needed initially for the treatment of ongoing and acute diseases. A subsequent dose may be higher than an earlier dose. To obtain the most efficacious results, depending on the disease or disorder, the antagonist is administered as close to the first sign, diagnosis, appearance, or occurrence of the disease or disorder as possible or during remissions of the disease or disorder.

The antagonist is administered by any suitable means, including parenteral, subcutaneous, intra-peritoneal, inhalational, intra-thecal, intra-articular, and intra-nasal, and, if desired for local immunosuppressive treatment, intralesional administration. Parenteral infusions include intramuscular, intravenous, intra-arterial, intraperitoneal, or subcutaneous administration. In addition, the antagonist may suitably be administered by pulse infusion, e.g., with declining doses of the antagonist. Preferably the dosing is given by injections, most preferably intravenous or subcutaneous injections, depending in part on whether the administration is brief or chronic.

Those of skill in the art will consider such factors as the mammal's age, level of activity, hormone balance, general health in determining the effective amount, which is tailored to the mammal, for example by beginning with a low dosage and titrating the dosage to determine the effective amount. By the studies described herein it has been discovered that increasing the concentration of beta glucan does not necessarily increase the inhibition of osteoclast activity, and may actually reduce inhibition of osteoclast activity. At 100 pg the effect is similar to the effect obtained with bisphosphonates, which in various forms are used as drugs to control osteoporosis.

The mammal may be re-treated with the antagonist/antibody, as by being given more than one exposure or set of doses, such as at least about two exposures of the antagonist/antibody, for example, from about 2 to 60 exposures, and more particularly about 2 to 40 exposures, most particularly, about 2 to 20 exposures. Such exposures may be administered at various intervals, such as, for example, about 24-28 weeks or 48-56 weeks or longer. Preferably, such exposures are administered at intervals each of about 24-26 weeks or about 38-42 weeks, or about 50-54 weeks. In one embodiment, each antagonist/antibody exposure is provided as a single dose of the antagonist/antibody. In an alternative embodiment, each antagonist/antibody exposure is provided as separate doses of the antibody. However, not every antagonist/antibody exposure need be provided as a single dose or as separate doses.

The preferred antagonist is an antibody, e.g. an antibody such as RITUXAN®, which is not conjugated to a cytotoxic agent. In the methods set forth herein, the B-cell surface marker or CD20 antibody may be a naked antibody or may be conjugated with another molecule such as being covalently linked to a bone-targeting agent. The preferred CD20 antibody herein is a chimeric, humanized, or human CD20 antibody, more preferably rituximab, a humanized 2H7 (e.g. comprising the variable domain sequences in SEQ ID Nos. 2 and 8, or comprising a variable heavy-chain domain with alteration N100A or D56A and N100A in SEQ ID NO:8 and a variable light-chain domain with alteration M32L, or S92A, or M32L and S92A in SEQ ID NO:2, or comprising the light-chain variable region (V_{L}) sequence of SEQ ID NO:30 and the heavy-chain variable region (V_{H}) sequence of SEQ ID NO:8, wherein the antibody further contains an amino acid substitution of D56A in VH-CDR2, and N100 in VH-CDR3 is substituted with Y or W, especially comprising the v511 light-chain sequence of SEQ ID NO:31 and the v511 heavy-chain sequence of SEQ ID NO:32), chimeric or humanized A20 antibody (Immunomedics), or HUMAX-CD20™ human CD20 antibody (Genmab). Still more preferred is rituximab or a humanized 2H7. In another aspect, the preferred antibody induces a major clinical response as defined above.

In further embodiment of all the methods herein, the mammal has never been previously treated with drug(s), such as an agent that treats osteoclast-associated disorders or immunosuppressive agent(s), to treat the bone disorder and/or has never been previously treated with an antagonist or antibody to a B-cell surface marker (e.g. never been previously treated with a CD20 antibody). In a still further aspect, the mammal may have had a relapse with the bone disorder or suffered other tissue damage such as kidney damage before being treated in any of the methods above, including after the initial or a later antibody set of doses. However, preferably, the mammal has not had such a relapse before at least the initial treatment.

Antagonists that bind to B-cell surface markers can be used wherever it is desired to stimulate the production of bone in both humans and non-human animals. Veterinary uses include use in domestic animals, including livestock and companion animals. Specific applications include, without limitation, fractures, including non-union fractures and fractures in patients with compromised healing, such as diabetics, alcoholics, and the aged; bone grafts; healing bone following radiation-induced osteonecrosis; implants, including joint replacements and dental implants; repair of bony defects arising from surgery, such as cranio-maxilofacial repair following tumor removal, surgical reconstruction following traumatic injury, repair of hereditary or other physical abnormalities, and promotion of bone healing in plastic surgery; treatment of periodontal disease and repair of other dental defects; treatment of bone defects following therapeutic treatment of bone cancers; increase in bone formation during distraction osteogenesis; treatment of joint injuries, including repair of cartilage and ligament; repair of joints that have been afflicted with osteoarthritis; tendon repair and re-attachment; treatment of osteoporosis (including age-related osteoporosis, post-menopausal osteoporosis, glutocorticoid-induced osteoporosis, and disuse osteoporosis) and other conditions characterized by increased bone loss or decreased bone formation; elevation of peak bone mass in pre-menopausal women; and use in the healing of connective tissues associated with dura mater.

In any of the methods herein, one may administer to the mammal along with the antagonist or antibody that binds a B-cell surface marker an effective amount of a second medicament (where the antagonist or antibody that binds a B-cell surface marker (e.g., the CD20 antibody) is a first medicament). The methods of this invention may also be used in conjunction with orthopedic devices such as spinal fusion cages, spinal fusion hardware, internal and external bone fixation devices, screws and pins. The second medicament may be one or more medicaments, and include, for example, an agent that treats osteoclast-associated disorders, a cytotoxic agent, an immunosuppressive agent, anti-pain agent, or any combination thereof. Other various therapies known to those skilled in the art may also be applied. The type of such second medicament depends on various factors, including the type of bone disorder, the severity of the bone disorder, the condition and age of the mammal, the type and dose of first medicament employed, etc.

Examples of such additional medicaments include an agent that treats osteoclast-associated disorders, a chemotherapeutic agent, an interferon class drug such as interferon-alpha (e.g., from Amarillo Biosciences, Inc.), IFN-β-1a (REBIF® and AVONEX®) or IFN-β-1b (BETASERON®), an oligopeptide such as glatiramer acetate (COPAXONE®), an agent blocking CD40-CD40 ligand, a cytotoxic or immunosuppressive agent (such as mitoxantrone (NOVANTRONE®), methotrexate, cyclophosphamide, chlorambucil, leflunomide, and azathioprine), intravenous immunoglobulin (gamma globulin), lymphocyte-depleting therapy (e.g., mitoxantrone, cyclophosphamide, CAMPATH™ antibodies, anti-CD4, cladribine, a polypeptide construct with at least two domains comprising a de-immunized, autoreactive antigen or its fragment that is specifically recognized by the Ig receptors of autoreactive B-cells (WO 2003/68822), total body irradiation, bone marrow transplantation, integrin antagonist or antibody (e.g., an LFA-1 antibody such as efalizumab/RAPTIVA® commercially available from Genentech, or an alpha 4 integrin antibody such as natalizumab/TYSABRI® available from Biogen Idec, or others as noted above), steroid such as corticosteroid (e.g., methylprednisolone such as SOLU-MEDROL™ methylprednisolone sodium succinate for injection, prednisone such as low-dose prednisone, dexamethasone, or glucocorticoid, e.g., via joint injection, including systemic corticosteroid therapy), non-lymphocyte-depleting immunosuppressive therapy (e.g., MMF or cyclosporine), cholesterol-lowering drug of the “statin” class (which includes cerivastatin (BAYCOL™), fluvastatin (LESCOL™), atorvastatin (LIPITOR™), lovastatin (MEVACOR™), pravastatin (PRAVACHOL™), and simvastatin (ZOCOR™)), estradiol, testosterone (optionally at elevated dosages; Stuve et al. Neurology 8:290-301 (2002)), androgen, hormone-replacement therapy, a TNF inhibitor such as an antibody to TNF-α, DMARD, NSAID, plasmapheresis or plasma exchange, trimethoprim-sulfamethoxazole (BACTRIM™, SEPTRA™), mycophenolate mofetil, H2-blockers or proton-pump inhibitors (during the use of potentially ulcerogenic immunosuppressive therapy), levothyroxine, cyclosporin A (e.g. SANDIMMUNE®), somatastatin analogue, cytokine, anti-metabolite, immunosuppressive agent, rehabilitative surgery, radioiodine, thyroidectomy, BAFF antagonist such as BAFF or BR3 antibodies or immunoadhesins, anti-CD40 receptor or anti-CD40 ligand (CD154), anti-IL-6 receptor antagonist/antibody, another B-cell surface antagonist or antibody such as a humanized 2H7 or other humanized or human CD20 antibody with rituximab, etc.

Preferred such medicaments are an agent that treats osteoclast-associated disorders, a chemotherapeutic agent, a cytotoxic agent, anti-integrin, gamma globulin, anti-CD4, cladribine, trimethoprimsulfamethoxazole, an H2-blocker, a proton-pump inhibitor, a corticosteroid, cyclosporine, cholesterol-lowering drug of the statin class, estradiol, testosterone, androgen, hormone-replacement drug, a TNF inhibitor, DMARD, NSAID (to treat, for example, musculoskeletal symptoms), levothyroxine, cyclosporin A, somatastatin analogue, BAFF antagonist such as BAFF antibody or BR3 antibody, especially a BAFF antibody, immunosuppressive agent, and another B-cell surface marker antibody, such as a combination of rituximab and a humanized 2H7 or other humanized CD20 antibody.

The more preferred such medicaments are an agent that treats osteoclast-associated disorders, such as a cytokine, an immunosuppressive agent, including an antibody against TNF-α, an antibody against CD40-CD40 ligand, and a BAFF antagonist such as a BAFF or BR3 antibody, a DMARD, a cytotoxic agent, an integrin antagonist, a NSAID, or a hormone, or a combination thereof. Immunosuppressants may be required, for example, for very active disease with major organ involvement, and include such agents as cyclophosphamide (CYTOXAN®), chlorambucil, leflunomide, MMF, azathioprine (IMURAN®), and methotrexate. BAFF antagonists may be useful in combination with the first medicament for efficacy.

Still more preferred are an agent that treats osteoclast-associated disorders, e.g., a cytokine such as IL-4, an immunosuppressive agent, or a combination thereof, most preferably an agent that treats osteoclast-associated disorders and/or an immunosuppressive agent, still most preferably, a bisphosphonate and/or methotrexate.

In one particularly preferred embodiment, the second medicament is or comprises one or more agents that treat osteoclast-associated disorders.

In a still further particularly preferred aspect, the second medicament is an immunosuppressive agent, more preferably cyclophosphamide, MMF, chlorambucil, azathioprine, leflunomide, or methotrexate, and preferably administered at least with the initial antibody doses. In one embodiment, azathioprine, methotrexate, or MMF are preferably used instead of cyclophosphamide for the maintenance of remission.

In a yet further preferred aspect, the second medicament is a combination of one or more agents that treat osteoclast-associated disorders and immunosuppressive agent.

All these second medicaments may be used in combination with each other or by themselves with the first medicament, so that the expression “second medicament” as used herein does not mean it is the only medicament besides the first medicament, respectively. Thus, the second medicament need not be one medicament, but may constitute or comprise more than one such drug.

These second medicaments as set forth herein are generally used in the same dosages and with administration routes as used hereinbefore or about from 1 to 99% of the heretofore-employed dosages. If such second medicaments are used at all, preferably, they are used in lower amounts than if the first medicament were not present, especially in subsequent dosings beyond the initial dosing with the first medicament, so as to eliminate or reduce side effects caused thereby. The combined administration includes co-administration, using separate formulations or a single pharmaceutical formulation, and consecutive administration in either order, wherein preferably there is a time period while both (or all) active agents simultaneously exert their biological activities.

For the re-treatment method herein, where a second medicament is administered in an effective amount with an antibody set of doses, it may be administered with any set of doses, for example, only with one set of doses, or with more than one set of doses. In one embodiment, the second medicament is administered with the initial set of doses. In another embodiment, the second medicament is administered with the initial and second set of doses. In a still further embodiment, the second medicament is administered with all sets of doses.

The combined administration of a second medicament includes co-administration (concurrent administration), using separate formulations or a single pharmaceutical formulation, and consecutive administration in either order, wherein preferably there is a time period while both (or all) active agents (medicaments) simultaneously exert their biological activities.

The antibody or antagonist herein is administered by any suitable means, including parenteral, topical, subcutaneous, intraperitoneal, intrapulmonary, intranasal, and/or intralesional administration. Parenteral infusions include intramuscular, intravenous (i.v.), intraarterial, intraperitoneal, or subcutaneous administration. Intrathecal administration is also contemplated (see, e.g., US 2002/0009444, Grillo-López, A., concerning intrathecal delivery of a CD20 antibody). In addition, the antibody or antagonist may suitably be administered by pulse infusion, e.g., with declining doses of the antibody or antagonist. Preferably, the dosing is given intravenously or subcutaneously, and more preferably by intravenous infusion(s).

If multiple sets of doses of antibody are provided, each set of doses may be provided using the same or a different administration means. In one embodiment, each set of doses is by intravenous administration. In another embodiment, each set of doses is given by subcutaneous administration. In yet another embodiment, the sets of doses are given by both intravenous and subcutaneous administration, and the antibodies may be the same or different.

A discussion of methods of producing, modifying, and formulating such antagonists and antibodies follows.

III. Production of Antagonists and Antibodies

The methods and articles of manufacture of the present invention use, or incorporate, an antagonist that binds to a B-cell surface marker. Accordingly, methods for generating such antagonists will be described here.

The B-cell surface marker to be used for production of, or screening for, antagonist(s) may be, e.g., a soluble form of the antigen or a portion thereof, containing the desired epitope. Alternatively, or additionally, cells expressing the B-cell surface marker at their cell surface can be used to generate, or screen for, antagonist(s). Other forms of the B-cell surface marker useful for generating antagonists will be apparent to those skilled in the art. Preferably, the B-cell surface marker is the CD19 or CD20 antigen.

While the preferred antagonist is an antibody, antagonists other than antibodies are contemplated herein. For example, the antagonist may comprise a small molecule antagonist optionally fused to, or conjugated with, a cytotoxic agent (such as those described herein). Libraries of small molecules may be screened against the B cell surface marker of interest herein in order to identify a small molecule that binds to that antigen. The small molecule may further be screened for its antagonistic properties and/or conjugated with a cytotoxic agent.

The antagonist may also be a peptide generated by rational design or by phage display (see, e.g., WO98/35036 published 13 Aug. 1998). In one embodiment, the molecule of choice may be a “CDR mimic” or antibody analogue designed based on the CDRs of an antibody. While such peptides may be antagonistic by themselves, the peptide may optionally be fused to a cytotoxic agent so as to add or enhance antagonistic properties of the peptide.

A description follows as to exemplary techniques for the production of the antibody antagonists used in accordance with the present invention.

(i) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler et al. Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al. Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al. Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies. Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-SEPHAROSE®, hydroxylapatite chromatography, gel electrophoresis, dialysis, or affinity chromatography.

The monoclonal antibodies may also be produced recombinantly. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al. Curr. Opinion in Immunol., 5:256-262 (1993) and Plückthun, Immunol. Revs., 130:151-188 (1992).

In a further embodiment, antibodies or antibody fragments can be isolated from antibody phage libraries generated using the techniques described in McCafferty et al. Nature, 348:552-554 (1990). Clackson et al. Nature, 352:624-628 (1991) and Marks et al. J. Mol. Biol., 222:581-597 (1991) describe the isolation of murine and human antibodies, respectively, using phage libraries. Subsequent publications describe the production of high affinity (nM range) human antibodies by chain shuffling (Marks et al. Bio/Technology, 10:779-783 (1992)), as well as combinatorial infection and in vivo recombination as a strategy for constructing very large phage libraries (Waterhouse et al. Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al. Proc. Natl. Acad. Sci. USA, 81:6851. (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. Preferably, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al. Nature, 321:522-525 (1986); Riechmann et al. Nature, 332:323-327 (1988); Verhoeyen et al. Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al. J. Immunol., 151:2296 (1993); Chothia et al. J. Mol. Biol., 196:901 (1987)). Another method uses a particular framework region derived from the consensus sequence of all human antibodies of a particular subgroup of light or heavy chains. The same framework may be used for several different humanized antibodies (Carter et al. Proc. Natl. Acad. Sci. USA, 89:4285 (1992); Presta et al. J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al. Proc. Natl. Acad. Sci. USA, 90:2551 (1993); Jakobovits et al. Nature, 362:255-258 (1993); Bruggermann et al. Year in Immuno., 7:33 (1993); and U.S. Pat. Nos. 5,591,669, 5,589,369 and 5,545,807.

Alternatively, phage display technology (McCafferty et al. Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al. Nature, 352.624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al. J. Mol. Biol. 222:581-597 (1991), or Griffith et al. EMBO J. 12:725-734 (1993). See, also, U.S. Pat. Nos. 5,565,332 and 5,573,905.

Human antibodies may also be generated by in vitro activated B cells (see U.S. Pat. Nos. 5,567,610 and 5,229,275).

(v) Antibody Fragments

Various techniques have been developed for the production of antibody fragments. Traditionally, these fragments were derived via proteolytic digestion of intact antibodies (see, e.g., Morimoto et al. Journal of Biochemical and Biophysical Methods 24:107-117 (1992) and Brennan et al. Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al. Bio/Technology 10: 163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody,” e.g., as described in U.S. Pat. No. 5,641,870 for example. Such linear antibody fragments may be monospecific or bispecific.

(vi) Bispecilic Antibodies

Bispecific antibodies are antibodies that have binding specificities for at least two different epitopes. Exemplary bispecific antibodies may bind to two different epitopes of the B cell surface marker. Other such antibodies may bind a first B cell marker and further bind a second B cell surface marker. Alternatively, an anti-B cell marker binding arm may be combined with an arm which binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the B cell. Bispecific antibodies may also be used to localize cytotoxic agents to the B cell. These antibodies possess a B cell marker-binding arm and an arm that binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂ bispecific antibodies).

Methods for making bispecific antibodies are known in the art. Traditional production of full-length bispecific antibodies is based on the co-expression of two immunoglobulin heavy-chain-light-chain pairs, where the two chains have different specificities (Millstein et al. Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al. EMBO J, 10:3655-3659 (1991).

According to a different approach, antibody variable domains with the desired binding specificities (antibody-antigen combining sites) are fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding, present in at least one of the fusions. DNAs encoding the immunoglobulin heavy-chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy-chain-light-chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al. Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al. Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al. J. Exp. Med., 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al. J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al. Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al. J. Immunol., 152:5368 (1994).

Antibodies with more than two valencies are contemplated. For example, trispecific antibodies can be prepared. Tutt et al. J. Immunol. 147: 60 (1991).

IV. Conjugates and Other Modifications of the Antagonist

The antagonist used in the methods or included in the articles of manufacture herein is optionally conjugated to another agent, such as a bone-targeting agent, so that delivery of, for example, systemically administered compositions of the present invention may be enhanced by conjugating CD20 antagonist to a targeting molecule. A “targeting molecule” is a molecule that binds to the tissue of interest. For example, bone-targeting molecules include tetracyclines, calcein, bisphosphonates, polyaspartic acid, polyglutamic acid, aminophosphosugars, peptides known to be associated with the mineral phase of bone (e.g., osteonectin, bone sialoprotein, and osteopontin), bone-specific antibodies, proteins with bone mineral or bone cell binding domains (e.g., calcitonin), and the like. See, for example, the disclosures of EP 512,844; EP 341,961; and Brinkley, Bioconjugate Chem. 3:2-13 (1992).

Conjugation will ordinarily be achieved through a covalent linkage, the precise nature of which will be determined by the targeting molecule and the linking site on the CD20 antagonist polypeptide. Typically, a non-peptidic agent is modified by the addition of a linker that allows conjugation to CD20 antagonist through its amino acid side chains, carbohydrate chains, or reactive groups introduced on CD20 antagonist by chemical modification. For example, a drug may be attached through the ε-amino group of a lysine residue, through a free α-amino group, by disulfide exchange to a cysteine residue, or by oxidation of the 1,2-diols in a carbohydrate chain with periodic acid to allow attachment of drugs containing various nucleophiles through a Schiff-base linkage. See, for example, U.S. Pat. No. 4,256,833.

Protein modifying agents include amine-reactive reagents (e.g., reactive esters, isothiocyantates, aldehydes, and sulfonyl halides), thiol-reactive reagents (e.g., haloacetyl derivatives and maleimides), and carboxylic acid- and aldehyde-reactive reagents. CD20 antagonist polypeptides can be covalently joined to peptidic agents through the use of bifunctional cross-linking reagents. Heterobifunctional reagents are more commonly used and permit the controlled coupling of two different proteins through the use of two different reactive moieties (e.g., amine-reactive plus thiol, iodoacetamide, or maleimide). The use of such linking agents is well known in the art. See, for example, Brinkley, supra, and U.S. Pat. No. 4,671,958. Peptidic linkers can also be employed. In the alternative, a CD20 antagonist polypeptide can be linked to a peptidic moiety through preparation of a fusion polypeptide.

Examples of further bifunctional protein coupling agents include N-succinimidyl-3-(2-pyridyldithiol)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

Alternatively, a fusion protein comprising the antagonist and agent may be made, e.g. by recombinant techniques or peptide synthesis.

Other modifications of the antagonist are contemplated herein. For example, the antagonist may be linked to one of a variety of nonproteinaceous polymers, e.g., polyethylene glycol, polypropylene glycol, polyoxyalkylenes, or copolymers of polyethylene glycol and polypropylene glycol.

The antagonists disclosed herein may also be formulated as liposomes. Liposomes containing the antagonist are prepared by methods known in the art, such as described in Epstein et al. Proc. Natl. Acad. Sci. USA, 82:3688 (1985); Hwang et al. Proc. Natl. Acad. Sci. USA, 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al. J. Biol. Chem. 257: 286-288 (1982) via a disulfide interchange reaction. A chemotherapeutic agent is optionally contained within the liposome. See Gabizon et al. J. National Cancer Inst. 81(19):1484 (1989).

Amino acid sequence modification(s) of protein or peptide antagonists described herein are contemplated. For example, it may be desirable to improve the binding affinity and/or other biological properties of the antagonist. Amino acid sequence variants of the antagonist are prepared by introducing appropriate nucleotide changes into the antagonist nucleic acid, or by peptide synthesis. Such modifications include, for example, deletions from, and/or insertions into and/or substitutions of, residues within the amino acid sequences of the antagonist. Any combination of deletion, insertion, and substitution is made to arrive at the final construct, provided that the final construct possesses the desired characteristics. The amino acid changes also may alter post-translational processes of the antagonist, such as changing the number or position of glycosylation sites.

A useful method for identification of certain residues or regions of the antagonist that are preferred locations for mutagenesis is called “alanine-scanning mutagenesis” as described by Cunningham and Wells Science, 244:1081-1085 (1989). Here, a residue or group of target residues are identified (e.g., charged residues such as Arg, Asp, His, Lys, and Glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antagonist variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antagonist with an N-terminal methionyl residue or the antagonist fused to a cytotoxic polypeptide. Other insertional variants of the antagonist molecule include the fusion to the NB or C-terminus of the antagonist of an enzyme, or a polypeptide that increases the serum half-life of the antagonist.

Another type of variant is an amino acid substitution variant. These variants have at least one amino acid residue in the antagonist molecule replaced by different residue. The sites of greatest interest for substitutional mutagenesis of antibody antagonists include the hypervariable regions, but FR alterations are also contemplated. Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in the biological properties of the antagonist are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: Norleucine, Met, Ala, Val, Leu, Ile;

(2) neutral hydrophilic: Cys, Ser, Thr;

(3) acidic: Asp, Glu;

(4) basic: Asn, Gin, His, Lys, Arg;

(5) residues that influence chain orientation: Gly, Pro; and

(6) aromatic: Trp, Tyr, Phe.

Non-conservative substitutions will entail exchanging a member of one of these classes for another class.

Any cysteine residue not involved in maintaining the proper conformation of the antagonist also may be substituted, generally with serine, to improve the oxidative stability of the molecule and prevent aberrant crosslinking. Conversely, cysteine bond(s) may be added to the antagonist to improve its stability (particularly where the antagonist is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine-scanning mutagenesis can be performed to identify hypervariable region residues contributing significantly to antigen binding. Alternatively, or in additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antagonist alters the original glycosylation pattern of the antagonist. By altering is meant deleting one or more carbohydrate moieties found in the antagonist, and/or adding one or more glycosylation sites that are not present in the antagonist.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antagonist is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antagonist (for O-linked glycosylation sites).

Nucleic acid molecules encoding amino acid sequence variants of the antagonist are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antagonist.

It may be desirable to modify the antagonist of the invention with respect to effector function, e.g. so as to enhance antigen-dependent cell-mediated cyotoxicity (ADCC) and/or complement dependent cytotoxicity (CDC) of the antagonist. This may be achieved by introducing one or more amino acid substitutions in an Fc region of an antibody antagonist. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al. J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

To increase the serum half life of the antagonist, one may incorporate a salvage receptor binding epitope into the antagonist (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG4) that is responsible for increasing the in vivo serum half-life of the IgG molecule.

V. Pharmaceutical Formulations

Therapeutic formulations of the antagonists used in accordance with the present invention are prepared for storage by mixing an antagonist having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Exemplary anti-CD20 antibody formulations are described in WO 1998/56418. This publication describes a liquid multidose formulation comprising 40 mg/mL rituximab, 25 mM acetate, 150 mM trehalose, 0.9% benzyl alcohol, 0.02% polysorbate 20 at pH 5.0 that has a minimum shelf life of two years storage at 2-8° C. Another anti-CD20 formulation of interest comprises 10 mg/mL rituximab in 9.0 mg/mL sodium chloride, 7.35 mg/mL sodium citrate dihydrate, 0.7 mg/mL polysorbate 80, and Sterile Water for Injection, pH 6.5.

Lyophilized formulations adapted for subcutaneous administration are described in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

The formulation herein may also contain more than one active compound (a second medicament as noted above) as necessary, preferably those with complementary activities that do not adversely affect each other. The type and effective amounts of such medicaments depend, for example, on the amount of antagonist present in the formulation, and clinical parameters of the mammals being treated. The preferred such medicaments are noted above.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release formulations may be prepared. Suitable examples of sustained-release preparations include semipermeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOTJ™ (injectable microspheres composed of lactic acid-glycolic acid copolymer and leuprolide acetate), and poly-D-(−)-3-hydroxybutyric acid.

Additional pharmaceutically acceptable delivery vehicles herein include biocompatible solid or semi-solid matrices, including powdered bone, ceramics, biodegradable and non-biodegradable synthetic polymers, and natural polymers; tissue adhesives (e.g., fibrin-based); aqueous polymeric gels; aqueous solutions; liposomes; and the like. Exemplary formulations and delivery vehicles are disclosed below. This disclosure is illustrative; those skilled in the art will readily recognize suitable alternatives, including derivatives of the specifically named materials and combinations of materials. Formulations may further include one or more additional growth factors, excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995.

CD20 antagonist can be delivered as a component of a tissue adhesive. Fibrin-based tissue adhesives are known in the art, and can be prepared from plasma or recombinant sources. Tissue adhesives comprise fibrinogen and factor XIII to which thrombin is added immediately before use to activate cross-linking. See, for example, U.S. Pat. Nos. 4,414,976; 4,427,650; and 4,928,603. The use of tissue adhesives may be particularly advantageous in the treatment of conditions where connective tissue must be repaired, such as torn ligaments or tendons. CD20 antagonist may also be combined with collagen-based adhesives. The collagen may be isolated from natural or recombinant sources.

Solid and semisolid matrices are preferred delivery vehicles for filling non-union fractures, cavities, and other bony defects. These matrices provide a space-filling substitute for the natural bone, and include bone substituting agents such as tricalcium phosphate, hydroxyapatite, combinations of tricalcium phosphate and hydroxyapatite, polymethylmethacrylate, aluminates and other ceramics, and demineralized freeze-dried cortical bone. Solid and semi-solid matrices can also be prepared from a variety of polymeric materials. Semi-solid matrices provide the advantage of maleability such that they can be shaped to provide a precise filling of a bony defect. Matrices may include other active or inert components. Of particular interest are those agents that promote tissue growth or infiltration. Agents that promote bone growth include bone morphogenic proteins (U.S. Pat. No. 4,761,471 and WO 90/11366), osteogenin (Sampath et al., Proc. Natl. Acad. Sci. USA 84: 7109-7113 (1987), and NaF (Tencer et al., J. Biomed. Mal. Res. 23: 571-589 (1989)).

Biodegradable, synthetic polymers include polyesters, polyorthoesters, polyanhydrides, polycarbonates, polyfumarates, polyhydroxybutyrate, vinyl polymers, and the like. Specific examples include, without limitation, polylactide, polyglycolide, polylactide/polyglycolide copolymers, polydioxanone, polyglycolide/trimethylene carbonate copolymers, polyacrylic acid, polymethacrylic acid, polyvinyl pyrrolidone, and polyvinyl alcohol. Such materials can be prepared in a variety shapes, including films, plates, pins, rods, screws, blocks, lattices, and the like for attachment to or insertion into bone. See, for example, U.S. Pat. No. 5,863,297; and WO 93/20859. These materials may further include a carrier such as albumin, a polyoxyethylenesorbitan detergent or glutamic acid. In principle, any substance that enhances polymer degradation, creates pores in the matrix or reduces adsorption of the growth factor(s) to the matrix can be used as a carrier. Polyoxyethylenesorbitan detergents that are useful as carriers include polyoxyethylenesorbitan monooleate, polyoxyethylenesorbitan monolaureate, polyoxyethylenesorbitan monopalmitate, polyoxy-ethylenesorbitan monostearate and polyoxyethylenesorbitan trioleate. Plasticizers can also be included.

In general, a film or device as described herein is applied to the bone at a site of injury. Application is generally by implantation into the bone or attachment to the surface using standard surgical procedures.

Biodegradable polymer films are particularly useful as coatings for prosthetic devices and surgical implants. Such films can, for example, be wrapped around the outer surfaces of surgical screws, rods, pins, plates and the like, or can themselves be rolled or otherwise formed into a variety of shapes. Implantable devices of this type are routinely used in orthopedic surgery. Films can also be used to coat bone filling materials, such as hydroxyapatite blocks, demineralized bone matrix plugs, collagen matrices, and the like. As used herein the term “copolymer” includes any polymer containing two or more types of monomer unit.

Degradation of the matrix and consequent release of growth factors therefrom can be modulated by adjusting such parameters as molecular weight, copolymer structure, copolymer ratio, matrix thickness, and porosity, and by including a carrier as disclosed above. PLA/PGA films, for example, are generally formulated to provide a ratio of PLA:PGA between 75:25 and 25:75, more commonly between 65:35 and 35:65. In general, an implant will be prepared using a copolymer having a molecular weight between 10,000 and 200,000 Daltons. In general, lower molecular weight copolymers will degrade more rapidly than higher molecular weight formulations; random copolymers are less crystalline and therefore degrade more quickly than other types of copolymers; and polymers of enantiomeric lactides are crystalline and therefore more resistant to degradation than their racemic counterparts.

Polymer matrices are prepared according to procedures known in the art. See, for example, U.S. Pat. No. 4,902,515; Gilding and Reed, Polymer 20: 1459-1464 (1979); and U.S. Pat. No. 3,773,919. For example, PLA/PGA copolymer implants are produced by combining the desired amount of PLA/PGA copolymer granules in a suitable solvent (e.g., chloroform or methylene chloride), pouring the resulting solution into a mold, and completely evaporating the solvent. In the alternative, PLA/PGA implants can be produced by compression molding, extrusion, or other known methods. To load the matrix, CD20 antagonist and a carrier are applied as powders or liquid solutions. For example, lyophilized CD20 antagonist and albumin may be uniformly dispersed over one surface of polymer film, and the film folded over. By repeated this process, a multi-layered “sandwich” of polymer and CD20 antagonist can be constructed. In the alternative, the proteins can be applied as aqueous solutions (e.g., in phosphate buffered saline or 0.1 M acetic acid), which are allowed to dry. Porous implants can be soaked in a solution of CD20 antagonist (optionally containing other components), and the liquid evaporated. CD20 antagonist can be worked into a malleable polymeric matrix after which the matrix is formed into the desired shape and cured at elevated temperature (e.g., 60-65° C.). Porous implants can be prepared by curing the matrix under vacuum.

The CD20 antagonist can also be delivered in combination with a biodegradable sponge, for example a gelatin, collagen, cellulose, or chitin sponge. Such sponges are known in the art. See, for example, U.S. Pat. No. 2,465,357; U.S. Pat. No. 4,271,070; and WO 90/13320. A solution of CD20 antagonist and, optionally, one or more additional therapeutic agents is injected into the sponge, and the sponge is air-dried at a temperature of 30-100° C. for a time sufficient to reduce the water content to below 50%, preferably below 10%.

Gels can also be used as delivery vehicles. The use of aqueous, polymeric gels for the delivery of growth factors is disclosed by, for example, U.S. Pat. Nos. 5,427,778; 5,770,228; 4,717,717; and 5,457,093. Gels comprise biocompatible, water soluble or water swellable polymers that form viscous solutions in water. Such polymers include, without limitation, polysaccharides, including methyl cellulose, hydroxypropyl cellulose, hydroxypropylmethyl cellulose, hydroxyethyl cellulose, dextrans, starch, chitosan, and alginic acid; glycosaminoglycans, including hyaluronic acid, chondroitin, chondroitin sulfates, heparin, and heparan sulfate; proteins, including collagen, gelatin, and fibronectin; and acrylamides, including polyacrylamide and polymethacrylamide. Gels are generally prepared with a viscosity of from 200 cps to 100,000 cps, more commonly about 1000 cps to 30,000 cps at room temperature, the latter range corresponding to about 0.25-10% hydroxyethyl cellulose in water. Higher viscosity gels are known in the art (e.g., U.S. Pat. No. 5,427,778). Viscosity can be adjusted by varying the concentration and/or length of the component polymer(s). Gels are prepared by combining the polymer with a suitable buffer, such as a low ionic strength citrate, phosphate, or acetate buffer at neutral or slightly acidic pH. A preservative (antimicrobial agent) such as methyl paraben, propyl paraben, benzyl alcohol, or the like, will generally be included. Following thorough mixing, the solution is sterilized by suitable means (e.g., autoclaving). The mixture is cooled, and filter-sterilized CD20 antagonist is added.

Alternative means for local delivery of CD20 antagonist include osmotic minipumps (e.g., ALZET® minipumps; Alza Corporation, Mountain View, Calif.); electrically charged dextran beads as disclosed in WO 92/03125; collagen-based delivery systems, such as disclosed in Ksander et al. (Ann. Surg. 211:288-294, 1990); and alginate-based systems as disclosed in Edelman et al. (Biomaterials, 12:619-626, 1991). Other methods known in the art for sustained local delivery in bone include porous coated metal protheses that can be impregnated with a therapeutic agent and solid plastic rods with therapeutic compositions incorporated within them.

The CD20 antagonist can be further used to treat osteoporosis by administering a therapeutically effective amount of CD20 antagonist to an individual. CD20 antagonist proteins can be tested in intact animals using an in vivo dosing assay. Prototypical dosing may be accomplished by subcutaneous, intraperitoneal or oral administration, and may be performed by injection, sustained release or other delivery techniques. The time period for administration of CD20 antagonist may vary (for instance, 28 days as well as 35 days may be appropriate).

The CD20 antagonist can be implanted in a mammalian body so that the CD20 antagonist is in contact with osteoblasts such that osteoblast proliferation occurs and bone growth is stimulated. For example, CD20 antagonist can be placed in a matrix in association with a bone morphogenic protein (BMP). The BMP induces the migration of mesenchymal osteoblast precursors to the site and further induces differentiation of the mesenchymal cells into osteoblasts. CD20 antagonist will then stimulate the further proliferation of the osteoblasts. A suitable matrix is made up of particles of porous materials. The pores must be of a dimension to permit progenitor cell migration and subsequent differentiation and proliferation, generally in the range of 70-850 μm, commonly from 150 μm to 420 μm. The matrix containing the CD20 antagonist can be molded into a shape encompassing a bone defect. Examples of matrix materials are particulate, demineralized, guanidine extracted, species-specific bone. Other potentially useful matrix materials include collagen, homopolymers and copolymers of glycolic acid and lactic acid, hydroxyapatite, tricalcium phosphate and other calcium phosphates. CD20 antagonist can be applied into a matrix at a sufficient concentration to promote the proliferation of osteoblasts, preferably at a concentration of at least 1 μg/ml of matrix. A solution of CD20 antagonist can also be injected directly into the site of a bone fracture to expedite healing of the fracture. Examples of BMPs and the use of matrices to produce are disclosed in WO 92/07073, WO 91/05802, U.S. Pat. No. 5,645,591 and U.S. Pat. No. 5,108,753.

The formulations to be used for in vivo administration must be sterile. This is readily accomplished by filtration through sterile filtration membranes.

Within another embodiment, the present invention provides methods for stimulating the growth and/or differentiation of bone-forming cells, or their precursors, in vitro. Using these methods, cells can be harvested from a patient, expanded ex vivo, and returned to the patient as generally disclosed above. Of particular interest is the growth and/or differentiation of bone marrow cells, which can be cultured in the presence of differentiation-stimulating agents to develop into, inter alia, osteoblasts, osteoclasts, and chondrocytes. Identification of differentiated cells within a primary culture is primarily phenotypic. For example, the phenotypic markers for osteoblasts include expression of alkaline phosphatase (Manduca et al., J. Bone Min. Res. 8:281 (1993)), type 1 collagen synthesis (Kurihara et al., Endocrinol. 118(3):940-947 (1986)), production of osteocalcin (Yoon et al., Biochem. 27:8521-8526 (1988)) and responsiveness to parathyroid hormone (Aubin et al., J. Cell Biol., 92:452-461 (1982)). Osteoblast cells are typically cultured at 37° C. in 5% CO₂ in a growth medium that includes a carbon source, a nitrogen source, essential amino acids, vitamins, minerals and growth factors generally supplied by fetal calf serum, variety of suitable media are known in the art. CD20 antagonist polypeptides are added to tissue culture media for these cell types at a concentration of about 10 μg/ml to about 1000 ng/ml. Those skilled in the art will recognize that CD20 antagonist proteins can be advantageously combined with other growth factors in culture media.

Bony defects may also be repaired using a gene-therapy approach wherein a polynucleotide encoding an antibody to a B-cell surface marker is administered to a patient. Gene delivery systems useful in this regard include adenovirus, adeno-associated virus, and naked DNA vectors. See, for example, U.S. Pat. No. 5,399,346; Mann et al., Cell 33:153 (1983); U.S. Pat. No. 4,650,764; U.S. Pat. No. 4,980,289; Markowitz et al., J. Virol. 62:1120 (1988); U.S. Pat. No. 5,124,263; WO 95/07358; and Kuo et al., Blood 82:845 (1993). Of particular interest is local infection of the affected tissue, such as local application of the vector to a periodontal pocket, fracture, joint, implant site, or site of prosthetic attachment.

In an alternative gene-therapy approach, US 2004/0126364 discloses delivery of polypeptides to mammals to promote bone tissue growth using living odontoprogenitor cells or osteoprogenitor cells (OPCs), a method applicable herein. The cells can be used for autologous or allogeneic cell transplants to serve as a cell-based platform to deliver the antibody herein in a site-specific, regulated manner. Systemic cell-based delivery is contemplated, as well as cell-based delivery of antibody that binds to a B-cell surface marker such as CD20 antibody at the site of an osteolytic lesion. The latter allows the antibody to be concentrated near an inflammatory site where inflammatory effector cells, e.g., macrophages, and osteolytic effector cells, e,g., osteoclast precursor cells, are located. The cells contain a genetically-engineered viral or non-viral plasmid vector generally containing a regulatable, inducible, osteoblast-specific promoter to direct expression of the antibody at specific sites of implantation in bone to inhibit osteolysis.

The cells for the purpose of these methods are typically committed to the osteoblastic lineage. For example, a bone stromal cell is isolated from autologous or allogeneic periodontal ligament and manipulated ex vivo prior to implantation into a recipient patient. The differentiated state of the bone marrow stromal cells or ligament derived cells is induced by culture in the presence of extracellular matrix (ECM) components such as MATRIGEL™ (Becton Dickenson) or other commercially available matrix preparation, preferably in the presence of one or more bone-morphogenetic proteins (BMP), such as BMP-2, -4, or -6. Induction of differentiation to progenitor cells is carried out before or after genetic manipulation of the cells; however, this step is preferably carried out before the differentiated cells are transduced with retroviral expression vectors containing genes encoding one or more therapeutic antibodies.

Differentiated progenitor cells have enhanced ability to build bone tissue, compared to undifferentiated stromal cells. OPCs or odontoprogenitor cells are distinguished from bone stromal cells (as well as fat, muscle, or cartilage cells or tissue) by the production of alkaline phosphatase, expression of osteocalcin, and expression of bone sialoprotein (in addition to the expression of dentin sialoprotein in the case of odontoprogenitors).

OPCs are isolated and expanded from stromal cells from bone marrow aspirates, and have been differentiated ex vivo in the presence of ECM. Autologous bone marrow stromal cells are expanded. The cells are optionally frozen and stored in liquid nitrogen for long periods of time before being differentiated and transduced. These “banked” autologous cells allow for multiple inoculations over a long period of time, which is beneficial since rheumatoid arthritis may persist for many years.

Since a clinical benefit is expected to be conferred by use of such cells, in further aspects, the invention herein contemplates a method of inhibiting osteolysis in a mammal, comprising introducing into said mammal an isolated odontoprogenitor or osteoprogenitor cell comprising a nucleic acid encoding an antibody that binds to a B-cell surface marker, preferably a CD20 antibody. The mammal is, e.g., a human patient that is suffering from or at risk of developing periodontitis or other bone disorders that may lead to bone loss, e.g., alveolar bone loss. The methods described herein are also applicable to veterinary use, e.g., to treat dogs, cats, horses.

The OPCs are genetically modified to contain a nucleic acid encoding the antibody herein, operably linked to a promoter that directs transcription of the nucleic acid to which it is linked preferentially in cells which have differentiated into osteoblasts. Retroviral expression vectors are typically constructed. Preferred promoters in such constructs are osteoblast-specific promoters such as those having an osteocalcin, bone sialoprotein, or dentin sialoprotein promoter sequence as described in the above identified US patent application, and/or a promoter to initiate transcription of the reverse transactivator tetracycline activator (rt TA) gene, which in turn regulates production of the therapeutic antibody. Additionally, a truncated fragment of such promoters, which functions to preferentially direct transcription in odontoprogenitor cells or OPCs (compared to other cell types) may be used. Preferably, the OPCs can be modified to increase the expression of the alpha-5 integrin receptor. This modification allows the cells to adhere to bone matrix proteins when implanted in vivo, which confers the added advantage that the OPCs may be inserted directly into osteolytic sites without prior encapsulation, e.g., in porous calcium phosphate ceramic cubes or other types of encapsulated devices. Expression of the nucleic acid encoding the antibody is preferably inducible. Osteoblast or odontoblast transcriptional regulatory DNA may be used to control expression of the antibody in a transcription unit. The regulatory sequence, e.g., a cis-acting cell-specific transcriptional regulatory element, may be positioned 5′ to the antibody-encoding nucleic acid sequence, in a transcription unit.

Expression of the antibody-encoding sequence may be regulated by contacting the cells with an antibiotic compound, such as, for example, tetracycline or a tetracycline analog (e.g., minocycline or doxycycline), and in one particular embodiment, tetracycline is given along with minocycline to the mammal, either together or separately. Such antibiotic compound is preferably administered systemically.

For example, tetracycline is systemically administered at least two days before periodontal surgery, e.g., and the time at which cells of the invention are implanted, and/or for at least two days after surgery and/or implantation. Expression of the antibody by the cells is turned on while the antibiotic is present in the tissue, i.e., while it is being administered to the cell implant recipient. Expression of the antibody decreases and ceases after administration of the antibiotic is stopped. Typically, an antibiotic administered 8-12 days prior to surgery and 8-12 days post-surgery. Similarly, antibiotics are administered before and after orthopedic surgery, e.g., surgery for cartilage removal from articulating joints or for removal of metastatic bone tumors (at which time the cells are implanted at or adjacent site to diseased tissue).

If the cell is an odontoprogenitor cell, said mammal may be suffering from or at risk of developing periodontitis, or alveolar bone loss due to periodontal disease. The cells may be implanted before, during, or after implantation of a dental orthopedic prosthesis. For the treatment of advanced periodontal disease, the cells may be administered locally to the periodontal ligament in the mandibular section of the jaw.

For treatment of bone disorders, the osteoprogenitor cells may be implanted into the bone marrow of a recipient mammal or into an articulating joint of the mammal. For example, the cells are administered intratibially or intrafemorally. The cells are implanted locally, e.g., at the site of bone loss or adjacent to such as site, e.g, in the bone marrow.

Methods of transplanting cells into the bone marrow of a mammal are well known in the art, e.g., as in U.S. Pat. No. 4,188,486. The dose of cells to be administered ranges from 1×10 cells to 1×10¹⁰ cells in volume suitable for the location of transplantation (e.g., a smaller volume is used for implantation into mandibular tissue or into the periodontal ligament compared to implantation into the bone marrow of the femur). Clinical protocols for such implantation procedures are known in the art. For example, a dose of 1×10⁸ cells per kg of body weight may be administered to femoral bone marrow. Repeated implants may be required for long-term diseases such as rheumatoid arthritis.

An agent that treats an osteoclast-associated disorder is optionally administered also with the antibody, such as, for example, IL-4, particularly recombinant human IL-4, or an inhibitor to TNF-α. In addition, other second medicaments may be optionally administered, such as NSAIDs or anti-pain agents, including aspirin, ibuprofen and indomethacin, as well as bisaryl COX-2 inhibitory compounds (e.g., as in U.S. Pat. No. 5,994,379) and (methylsulfonyl)-phenyl-2-(5H)-furanones (e.g., as in U.S. Pat. No. 6,020,343). More preferably, IL-4 or an inhibitor to TNF-α is additionally administered.

The isolated genetically-modified OPCs are used to treat individuals suffering from or at risk of developing a bone loss disorder such as rheumatoid arthritis, osteoporosis, periapical or endochondral bone loss, artificial joint particle-induced osteolysis, bone fracture or deficiency, primary or secondary hyperparathyroidism, metastatic bone disease, osteolytic bone disease, post-plastic surgery, post-prosthetic joint surgery. More preferably, such mammal may be suffering from or at risk of developing rheumatoid arthritis or periapical or endochondral bone loss, artificial joint particle-induced osteolysis, or osteolytic bone metastases. Thus, for example, the OPCs, engineered to secrete the antibodies herein, may be implanted in patients who are undergoing revision of an artificial joint replacement due to the development of implant-induced osteolysis, as well as in patients suffering bone loss due to rheumatoid arthritis and in the oral cavity due to severe periodontal disease.

VI. Articles of Manufacture

In another embodiment of the invention, articles of manufacture containing materials useful for the treatment of a bone disorder described above are provided. In one aspect, the article of manufacture comprises (a) a container comprising an antagonist that binds to a B-cell surface marker (e.g., an antibody that so binds, including a CD20 antibody) (preferably the container comprises the antagonist or antibody and a pharmaceutically acceptable carrier or diluent within the container); and (b) a package insert with instructions for treating a bone disorder in a mammal, such as a patient, wherein the instructions indicate that the antagonist or antibody is administered in an effective amount, such as, for example, a dose of the antagonist or antibody of about 400 mg to 1.3 grams at a frequency of one to four doses is administered to the patient within a period of about one month.

In a preferred embodiment, the article of manufacture herein further comprises a container comprising a second medicament, wherein the antagonist or antibody is a first medicament. This article further comprises instructions on the package insert for treating the patient with the second medicament, in an effective amount. The second medicament may be any of those set forth above, with an exemplary second medicament being an agent that treats osteoclast-associated disorders, an immunosuppressive agent, a cytotoxic agent, an integrin antagonist, or a hormone. The preferred second medicaments are those preferred as set forth above, and most preferred is an agent that treats osteoclast-associated disorders or an immunosuppressive agent or both.

In another aspect, the invention provides an article of manufacture comprising: (a) a container comprising an antibody that binds to a B-cell surface marker (e.g., a CD20 antibody) (preferably the container comprises the antibody and a pharmaceutically acceptable carrier or diluent within the container); and (b) a package insert with instructions for treating a bone disorder in a mammal, wherein the instructions indicate that an amount of the antibody is administered to the mammal that is effective to provide an initial antibody set of doses followed by a second antibody set of doses, wherein the second exposure is not provided until from about 16 to 54 weeks from the initial set of doses.

Preferably, such package insert is provided with instructions for treating a bone disorder in a mammal, wherein the instructions indicate that an amount of the antibody is administered to the mammal that is effective to provide an initial antibody exposure of about 0.5 to 4 grams followed by a second antibody exposure of about 0.5 to 4 grams, wherein the second exposure is not provided until from about 16 to 54 weeks from the initial exposure and each of the antibody exposures is provided to the mammal as about one to four doses, preferably as a single dose or as two to four separate doses of antibody.

In a preferred embodiment of this inventive aspect, the article of manufacture herein further comprises a container comprising a second medicament, wherein the antibody is a first medicament, and which article further comprises instructions on the package insert for treating the mammal with the second medicament, in an effective amount. The second medicament may be any of those set forth above, with an exemplary second medicament being an agent that treats osteoclast-associated disorders, an immunosuppressive agent, a cytotoxic agent, an integrin antagonist, or a hormone, most preferably an agent that treats osteoclast-associated disorders or an immunosuppressive agent, or both.

In all of these aspects, the package insert is on or associated with the container. Suitable containers include, for example, bottles, vials, syringes, etc. The containers may be formed from a variety of materials such as glass or plastic. The container holds or contains a composition that is effective for treating the bone disorder and may have a sterile access port (for example the container may be an intravenous solution bag or a vial having a stopper pierceable by a hypodermic injection needle). At least one active agent in the composition is the antagonist or antibody. The label or package insert indicates that the composition is used for treating a mammal such as a patient eligible for treatment, e.g., one having or predisposed to a bone disease such as those listed herein, with specific guidance regarding dosing amounts and intervals of antagonist or antibody and any other medicament being provided. The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

Further details of the invention are illustrated by the following non-limiting Examples. The disclosures of all citations in the specification are expressly incorporated herein by reference.

### EXAMPLE 1

### Serum Biochemical Markers Correlate with Response to Treatment of Rheumatoid Arthritis with Rituximab

Background:

Targeting B cells with rituximab (RTX; Rituxan®/MabThera®) has been found to provide a new approach for the treatment of rheumatoid arthritis (RA). A randomized controlled trial in patients with active rheumatoid arthritis despite methotrexate showed that a single course of two rituximab infusions, alone or in combination with cyclophosphamide or continued methotrexate, provided significant improvement in disease activity at week 24 (Edwards et al., N. Engl. J. Med. (2004), supra; Pavelka et al., supra) and week 48 (Edwards et al., N. Eng. J. Med. (2004), supra), which was sustained in some patients for two years (Emery et al., (2004), supra). However, little is known of its mechanism of action to treat RA or its effects on various biological markers in RA patients. In particular, it is not known whether a B cell-depleting therapy such as rituximab will have an effect on the activity of osteoclasts.

Purpose:

To further understand the effects of rituximab on serum biomarkers and whether such effect is consistent with clinical findings, a number of serological markers of inflammation and bone turnover were investigated.

Methods:

Serum samples were analyzed from RA+ patients who received either methotrexate (MTX) alone or MTX in combination with a single course of rituximab as part of a Phase IIa study of rituximab in RA to be reported elsewhere. The following markers were measured at baseline and Week 24: anti-CCP, CRP, S100, SAA, PINP and Osteocalcin (OC).

Results:

The primary endpoint of ACR50 response at 24 weeks demonstrated that patients receiving two infusions of rituximab two weeks apart with ongoing MTX achieved significantly higher clinical responses than those receiving MTX alone. Biomarker data were analyzed using a non-parametric approach by comparing the percentage changes at 24-weeks from baseline in MTX group with the RTX+MTX group. The median percent change from baseline was plotted using the following formula:
Pcfb1(marker)=((slope_{—}lr*study time at last visit)/intercept_{—}lr)*100

where slope_lr=slope of linear regression for each patient
intercept_lr=intercept of linear regression for each patient.

The clinical response observed in the RTX+MTX group at 24-weeks was also accompanied by significant changes in the serum levels of the markers analysed. The results are shown in Table 2.

TABLE 2

| | Median % change from baseline at 24 weeks | | |
|---|---|---|---|
| | MTX | Rituximab + M | |
| | (n = 40) | TX (n = 40) | P-value * |
| Anti-CCP¹ | −21.03 | −45.78 | 0.0012 |
| CRP² | −22.21 | −59.07 | 0.0087 |
| S100³ | −14.31 | −52.73 | 0.0004 |
| SAA⁴ | −10.55 | −77.02 | <0.0001 |
| P-1NP⁵ | −11.78 | 13.64 | 0.0119 |
| OC⁶ | 1.94 | 28.76 | 0.0143 |

* P-value from 2-sided Wilcoxon Rank Sum Test
¹Anti-CCP is anti-citrullinated peptide antibody
²CRP is C-reactive protein
³S100 is S100 A8/9, which is Ca⁺⁺ binding calgranulin protein in phagocytes
⁴SAA is serum amyloid A
⁵P-1NP is procollagen type 1 N-terminal propeptide
⁶OC is osteocalcin

Conclusions:

All the markers analysed showed a significant change at 24 weeks in the RTX+MTX group compared to MTX alone. In particular, the change in anti-CCP, CRP, S100 and SAA serum levels suggests that a single, short course with rituximab has a profound effect on markers of inflammation and autoantibodies, which is consistent with the substantial improvement on signs and symptoms observed. Total immunoglobulin levels were not affected significantly by treatment with rituximab. In addition, OC and PINP serum levels increased significantly in the RTX+MTX group compared with the MTX group, suggesting that the effect of rituximab on the signs and symptoms of RA is complemented by a positive effect on biomarkers of bone turnover, indicating an effect of rituximab on bone density and/or structural damage.

### EXAMPLE 2

### Bone Remodeling in Animals

The activity of Rituximab may be demonstrated in an animal model of bone remodeling. Thus, Sprague-Dawley rats are weight-matched and divided into seven groups, with ten animals in each group. Included is a baseline control group of rats sacrificed at the start of the study, a control group administered vehicle only, a PBS-treated control group, and a positive control group administered a compound known to promote bone growth. Three dosage levels of rituximab are administered to the remaining three groups.

Rituximab, positive control compound, PBS, or vehicle alone is administered subcutaneously once per day for 35 days. All animals are injected with calcein nine days and two days before sacrifice (two injections of calcein administered each designated day). Weekly body weights are determined. At the end of the 35-day cycle, the animals are weighed and bled by orbital or cardiac puncture. Serum calcium, phosphate, osteocalcin, and CBCs are determined. Both leg bones (femur and tibia) and lumbar vertebrae are removed, cleaned of adhering soft tissue, and stored in 70% ethanol for evaluation. The effect of rituximab on bone remodeling is performed by peripheral quantitative computed tomography (pQCT; Ferretti, Bone 17:353S-364S (1995)), dual energy X-ray absorptiometry (DEXA; Laval-Jeantet et al., Calcif Tissue Intl. 56:14-18 (1995); Casez et al., Bone and Mineral 26:61-68 (1994) and/or histomorphometry.

Administration of rituximab in vivo is expected to inhibit osteoclastogenesis and associated bone resorption and block the pathological increase in osteoclast numbers and activity seen in animal models that mimic osteopenic disorders in humans. Thus, it is expected that rituximab, or humanized 2H7, will be effective in enhancing bone remodeling over the controls in this model.

### EXAMPLE 3

### Clinical Study of Rituximab in Osteoporosis

Patients diagnosed with osteoporosis may be treated with rituximab. The patients treated will be selected so as not to have a B-cell malignancy.

Rituximab is administered intravenously (IV) to the patients according to any of the following dosing schedules:

(A) 50 mg/m² IV day 1

150 mg/m² IV on days 8, 15 & 22

(B) 150 mg/m² IV day 1

375 mg/m² IV on days 8, 15 & 22

(C) 375 mg/m² IV on days 1, 8, 15 & 22

(D) 1 g IV days 1 & 15

Further adjunct therapies (such as an agent that treats osteoclast-associated disorders, as noted above) may be combined with the rituximab therapy, but preferably the patients are treated with rituximab as a single-agent throughout the course of therapy. A control is administered to a separate group of patients using the placebo (solution of rituximab formulation without rituximab).

Overall response rate is determined based upon an improvement in bone osteoblasts as determined by standard chemical parameters. In addition, bone densitometry and assessment of fracture rate are employed as endpoints in addition to the biomarkers, such as set forth in Example 1. It is expected that administration of rituximab, or humanized 2H7, will improve bone osteoblasts as determined by biomarkers and improve bone densitometry and decrease fracture rate in the patients treated as described above versus the placebo control.

### EXAMPLE 4

### Identification of Osteoclasts Formed In Vitro

TRAP refers to tartrate-resistant acid phosphatase that identifies osteoclast-like cells. Rituximab can be used as a positive control in the TRAP assay.

Cytochemical staining for TRAP is widely used for identifying osteoclasts in vivo and in vitro. In this test, naphthol AS-MX phosphate 5 mg (Sigma, St. Louis, Mo.) is resolved in 0.5 ml of N,N-dimethylformamide (Wako). Thirty milligrams of fast red violet LB salt (Sigma) and 50 ml of 0.1 M sodium acetate buffer (pH 5.0) containing 50 mM sodium tartrate are added to the mixture (the TRAP-staining solution). Cells are fixed with 3.7% (v/v) formaldehyde in Ca²⁻ and Mg²⁺-free phosphate-buffered saline for 10 min, fixed again with ethanol-acetone (50:50, v:v) for 1 min, and incubated with the TRAP-staining solution for 10 min. at room temperature. TRAP-positive osteoclasts appear as red cells. The incubation period should not exceed 10 min. since cells other than osteoclasts become weakly positive with time. After staining, cells are washed with distilled water, and TRAP-positive multinucleated cells having three or more nuclei are counted as osteoclasts under a microscope (Nicholson et al., J. Clin. Invest. 78: 355 (1986)). It is expected that rituximab or humanized 2H7 will be effective as a positive control in this TRAP assay.

### EXAMPLE 5

### CD20 Antibody for Periodontal Disease

Rituximab may be used to regenerate bone and ligament lost to periodontal disease. In this model teeth showing 20% to 80% reduction of surrounding jaw bone are scaled, then a full-thickness gingival flap is made by an incision to expose the jaw bone and tooth root. The root is planed to remove bacterial plaque and calculus. Rituximab is applied to the periodontal pocket in a 2.5% HPMC gel at a dose of 100 g per tooth, or alternatively a solution of rituximab in 100 mM sodium acetate buffer, pH 6.0 is added to powdered bone to provide a dosage of 100 g rituximab per tooth. The material is thoroughly mixed and applied to the exposed periodontal pocket. In both cases, the gingival flap is then closed and held in place by suturing. It is expected that surrounding jaw bone will be at least partially regenerated in this periodontitis model using rituximab, or humanized 2H7, versus a control with placebo rather than rituximab.

### EXAMPLE 6

### Tooth Implants

The activity of rituximab may be demonstrated in this tooth implant model. Polylactic acid-polyglycolic acid films (50:50) are solvent cast by dissolving about 340 mg of polymer granules (Polysciences, Warrington, Pa.) in 10 ml chloroform at room temperature and allowing the solvent to evaporate completely in a slow air flow hood at room temperature. The films are 10 μm thick. Each is cut into a 80 mm×40 mm sheet, resulting in a remaining polymer mass of about 270-290 mg. A solution of rituximab or humanized 2H7 and rabbit serum albumin is dispersed on the films, and the liquid is allowed to evaporate. The films are then rolled around 0.9-mm diameter Kirschner wires to provide implants of 1.5- or 3.0-mm diameter and sterilized using cold ethylene oxide gas. These implants are placed into rats, along with implants having a placebo rather than the CD20 antibody. It is expected that bone loss will be prevented in this implant model using rituximab or humanized 2H7 versus the control implant.

### EXAMPLE 7

### Prevention of Bone Loss in OVX Animals

The activity of rituximab may be demonstrated in acute ovariectomized animals using an in vivo dosing assay with an estrogen-treated group as control. In this prevention model Sprague-Dawley rats are weight-matched and divided into eight groups. This includes a baseline control group of rats sacrificed at the initiation of the study; three control groups (sham ovariectomized (sham OVX)+vehicle only; ovariectomized (OVX)+vehicle only; PBS-treated OVX); and a control OVX group that is administered estrogen. Three dosage levels of rituximab are administered to the remaining three groups of OVX animals. Since ovariectomy (OVX) induces hyperphagia, all OVX animals are pair-fed with sham OVX animals throughout the 35-day study.

Rituximab, positive control compound, PBS, or vehicle alone is administered subcutaneously once per day for 35 days. Alternatively, rituximab is formulated in implantable pellets that are implanted for 35 days. All animals, including sham OVX/vehicle and OVX/vehicle groups, are injected intraperitoneally with calcein nine days and two days before sacrifice (two injections of calcein administered each designated day, to ensure proper labeling of newly formed bone). Weekly body weights are determined. At the end of the 35-day cycle, the animals' blood and tissues are processed as described above. It is expected that rituximab, or humanized 2H7, will be effective versus the control groups in preventing bone loss in this model.

### EXAMPLE 8

### Bone Effects in Chronic OVX Animals

The activity of rituximab may be demonstrated in chronic OVX animals. In this model several Sprague-Dawley rats are subjected to sham surgery (sham OVX) or ovariectomy (OVX) at time 0, and 10 rats are sacrificed to serve as baseline controls. Body weights are recorded weekly. After 6 weeks of bone depletion, 10 sham OVX and 10 OVX rats are randomly selected for sacrifice as depletion period controls. Of the remaining animals, 10 sham OVX and 10 OVX rats are used as placebo-treated controls. The remaining OVX animals are treated with 3 to 5 doses of rituximab for a period of 5 weeks. As a positive control, a group of OVX rats is treated with an agent such as PTH, a known anabolic agent in this model (Kimmel et al. Endocrinology 132:1577-1584 (1993)). The femurs, tibiae and lumbar vertebrae 1 to 4 are excised and collected to determine effects on bone formation. The proximal left and right tibiae are used for pQCT measurements, cancellous bone mineral density (BMD) (gravimetric determination), and histology, while the midshaft of each tibiae is subjected to cortical BMD or histology. The femurs are prepared for pQCT scanning of the midshaft prior to biomechanical testing. With respect to lumbar vertebrae (LV), LV2 are processed for BMD; LV3 are prepared for undecalcified bone histology; and LV4 are processed for mechanical testing. It is expected that the OVX animals treated with rituximab or humanized 2H7 will show improvement in one or more of these tests of femurs, tibiae and lumbar vertebrae 1 to 4 over the placebo-treated controls in enhancing bone formation.

### EXAMPLE 9

### Inhibition of Alveolar Bone Loss by Cell-Delivered CD20 Antibody

The osteoclast is responsible for mediating excessive bone resorption during progressive periodontitis. Rituximab is expected to inhibit osteoclast differentiation and function. In this animal model, autologous cells are engineered to express rituximab and permanently implanted at sites of inflammation, e.g., in the mandible, in soft tissue adjacent to affected teeth, or in the periodontal ligament, using methods known in the art.

Periodontal disease is induced in C3H mice by repeated injections of LPS derived from the clinically-relevant microorganism Porphyromonas gingivalis, an art recognized model of periodontal disease. Mice with periodontal disease are treated using the C₃H₁₀T1/2 murine fibroblast line genetically engineered to produce rituximab in a regulatable manner. Production of rituximab is regulated by providing antibiotic orally, e.g., in the drinking water. Cells are implanted locally, at sites of bone resorption, thereby bypassing the need for either systemic administration or repetitive local injections of a bioactive molecule. Optionally, antibiotics are placed in the periodontal pocket following implantation of cells for periodontal disease. This cell-based approach for local delivery of rituximab utilizes tissue engineering to inhibit resorption of alveolar bone.

The murine Moloney retroviral vectors used herein are well characterized and are non-immunogenic in humans or mice.

Standard in situ hybridization (ISH) is used to detect rituximab production as well as characterize the osteoclast phenotype in cells that have populated mandibular bone or other bone tissue of cell implant recipients.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for treating a bone disorder in a mammal comprising administering to the mammal an effective amount of a CD20 antibody.

2. The method of claim 1 wherein the antibody is a chimeric, human, or humanized antibody.

3. The method of claim 1 wherein the antibody comprises rituximab.

4. The method of claim 1 wherein the antibody is a humanized 2H7 comprising the variable domain sequences in SEQ ID Nos. 2 and 8.

5. The method of claim 1 wherein the antibody is a humanized 2H7 comprising a variable heavy-chain domain with alteration(s) N100A or D56A,N100A in SEQ ID NO:8 and a variable light-chain domain with alteration(s) M32L, S92A, or M32L,S92A in SEQ ID NO:2.

6. The method of claim 1 wherein the antibody is a humanized 2H7 comprising the light-chain variable region (V_{L}) sequence of SEQ ID NO:30 and the heavy-chain variable region (V_{H}) sequence of SEQ ID NO:8, wherein the antibody further contains an amino acid substitution of D56A in VH-CDR2, and N 100 in VII-CDR3 is substituted with Y or W.

7. The method of claim 6 wherein the antibody comprises the v511 light-chain sequence of SEQ ID NO:31 and the v511 heavy-chain sequence of SEQ ID NO:32.

8. The method of claim 1 wherein the antibody is a naked antibody.

9. The method of claim 1 wherein the antibody is conjugated with another molecule.

10. The method of claim 9 wherein the antibody is covalently linked to a bone-targeting agent.

11. The method of claim 1 wherein the antibody induces a major clinical response upon administration to the mammal.

12. The method of claim 1 wherein the antibody is administered in a dose of about 400 mg to 1.3 grams at a frequency of about one to four doses within a period of about one month.

13. The method of claim 12 wherein each dose is about 500 mg to 1.2 grams.

14. The method of claim 12 wherein each dose is about 750 mg to 1.1 grams.

15. The method of claim 12 wherein the antibody is administered in two to four doses.

16. The method of claim 12 wherein the antibody is administered in two to three doses.

17. The method of claim 12 wherein the antibody is administered within a period of about 2 to 3 weeks.

18. The method of claim 17 wherein the period is about two weeks.

19. The method of claim 1 wherein the mammal is human.

20. The method of claim 1 wherein the antibody is locally administered at a joint.

21. The method of claim 1 wherein the antibody is locally administered at a site of a bony defect.

22. The method of claim 21 wherein the bony defect is a fracture, bone graft site, implant site, or periodontal pocket.

23. The method of claim 1 wherein the antibody is administered systemically.

24. The method of claim 1 wherein the antibody is administered intravenously.

25. The method of claim 1 wherein the antibody is administered subcutaneously.

26. The method of claim 1 wherein a second medicament is administered in an effective amount, wherein the CD20 antibody is a first medicament.

27. The method of claim 26 wherein the second medicament is more than one medicament.

28. The method of claim 26 wherein the second medicament is an agent that treats osteoclast-associated disorders, an immunosuppressive agent a disease-modifying anti-rheumatic drug (DMARD), a cytotoxic agent, an integrin antagonist, a non-steroidal anti-inflammatory drug (NSAID), a hormone, or a combination thereof.

29. The method of claim 28 wherein the second medicament is an agent that treats osteoclast-associated disorders or an immunosuppressive agent, or both.

30. The method of claim 29 wherein the second medicament is an immunosuppressive agent.

31. The method of claim 30 wherein the immunosuppressive agent is cyclophosphamide, chlorambucil, leflunomide, azathioprine, or methotrexate.

32. The method of claim 31 wherein the immunosuppressive agent is cyclophosphamide or methotrexate.

33. The method of claim 29 wherein the second medicament is an agent that treats osteoclast-associated disorders.

34. The method of claim 33 wherein the agent is an osteoprotegerin, an interleukin, a MMP inhibitor, a beta glucan, an integrin antagonist, calcitonin, a proton pump inhibitor, a protease inhibitor, a bisphosphonate, insulin-like growth factor-1, platelet-derived growth factor, epidermal growth factor, an inhibitor of transforming growth factor-alpha, transforming growth factor-beta, a bone morphogenetic protein, parathyroid hormone, a fibroblast growth factor, Vitamin D, calcium, fluoride, magnesium, boron, vitronectin, plasminogen-activator inhibitor, or a protease inhibitor.

35. The method of claim 34 wherein the agent is a cytokine or bisphosphonate.

36. The method of claim 33 wherein the agent is administered in lower amounts than are used if the CD20 antibody is not administered to a mammal treated with the agent.

37. The method of claim 1 wherein the mammal has never been previously treated with a CD20 antibody.

38. The method of claim 1 wherein the bone disorder is osteoporosis, an osteoporotic fracture, focal bone loss, a bone defect, childhood idiopathic bone loss, alveolar bone loss, mandibular bone loss, alveolar bone loss, bone loss associated with periodontitis, bone loss associated with an autoimmune disease, or bone disease in multiple myeloma, macroglulinemia or monoclonal gammopathy.

39. The method of claim 38 wherein the bone disorder is focal bone loss, bone disease in multiple myeloma, macroglulinemia or monoclonal gammopathy, bone loss associated with an autoimmune disease, rheumatoid arthritis, or osteoporosis.

40. The method of claim 39 wherein the bone disorder is bone loss associated with rheumatoid arthritis or secondary osteoporosis.

41. The method of claim 39 wherein the bone disorder is focal bone loss.

42. The method of claim 1 wherein the amount of the CD20 antibody is effective to prevent erosive bone disease in inflammatory arthritides.

43. The method of claim 42 wherein the inflammatory arthritides is rheumatoid arthritis.

44. The method of claim 1 wherein the bone disorder is not associated with rheumatoid arthritis or a risk of developing rheumatoid arthritis.

45. The method of claim 1 wherein the antibody is administered in a delivery vehicle.

46. The method of claim 45 wherein the delivery vehicle is powdered bone, tricalcium phosphate, hydroxyapatite, polymethacrylate, a biodegradable polyester, an aqueous polymeric gel, or a fibrin sealant.

47. A method for treating a bone disorder in a mammal comprising administering to the mammal an effective amount of an antibody that binds to a B-cell surface marker.

48. A method for treating a bone disorder in a mammal comprising administering to the mammal an effective amount of an antagonist that binds to a B-cell surface marker.

49. An article of manufacture comprising:
i. a container comprising a CD20 antibody; and
ii. a package insert with instructions for treating a bone disorder in a mammal, wherein the instructions indicate that an effective amount of the CD20 antibody is administered to the mammal.

50. The article of claim 49 further comprising a container comprising a second medicament, wherein the CD20 antibody is a first medicament, further comprising instructions on the package insert for treating the mammal with the second medicament.

51. The article of claim 50 wherein the second medicament is an agent that treats osteoclast-associated disorders, an immunosuppressive agent, a cytotoxic agent, an integrin antagonist, or a hormone.

52. The article of claim 50 wherein the second medicament is an agent that treats osteoclast-associated disorders or an immunosuppressive agent, or both.

53. A method of inhibiting osteolysis in a mammal, comprising introducing into said mammal an isolated odontoprogenitor or osteoprogenitor cell comprising a nucleic acid encoding an antibody that binds to a B-cell surface marker.

54. The method of claim 53 wherein said cell is an odontoprogenitor cell.

55. The method of claim 54 wherein said mammal is suffering from or at risk of developing periodontitis.

56. The method of claim 54 wherein said mammal is suffering from or at risk of developing alveolar bone loss due to periodontal disease.

57. The method of claim 54 wherein said cell is administered to the periodontal ligament in the mandibular section of the jaw.

58. The method of claim 53 wherein said cell is an osteoprogenitor cell.

59. The method of claim 58 wherein said cell is implanted into an articulating joint of said mammal.

60. The method of claim 58 wherein said cell is administered intratibially.

61. The method of claim 58 wherein said cell is administered intrafemorally.

62. The method of claim 53 wherein expression of said antibody is regulated by an antibiotic compound.

63. The method of claim 62 wherein said antibiotic compound is tetracycline or a tetracycline analogue.

64. The method of claim 63 further comprising administering minocycline to said mammal.

65. The method of claim 62 wherein said antibiotic compound is administered systemically.

66. The method of claim 53 further comprising administering an agent that treats an osteoclast-associated disorder.

67. The method of claim 66 wherein the agent is interleukin-4 or an inhibitor of tumor necrosis factor-alpha.

68. The method of claim 53 wherein said mammal is suffering from or at risk of developing rheumatoid arthritis.

69. The method of claim 53 wherein said mammal is not suffering from or at risk of developing rheumatoid arthritis.

70. The method of claim 53 wherein said mammal is suffering from or at risk of developing periapical or endochondral bone loss, artificial joint particle-induced osteolysis, or osteolytic bone metastases.

71. The method of claim 53 wherein the antibody is a CD20 antibody.


SEQUENCE LISTING (32 sequences)

SEQ ID NO: 1 - protein, 107 aa, Mus musculus
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKR

SEQ ID NO: 2 - protein, 107 aa, Artificial
  humanized 2H7 light chain variable domain
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 3 - protein, 108 aa, Homo sapiens
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIKR

SEQ ID NO: 4 - protein, 10 aa, Artificial
  2H7 CDR1
RASSSVSYMH

SEQ ID NO: 5 - protein, 7 aa, Artificial
  2H7 CDR2
APSNLAS

SEQ ID NO: 6 - protein, 9 aa, Artificial
  2H7 CDR3
QQWSFNPPT

SEQ ID NO: 7 - protein, 122 aa, Mus musculus
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSS

SEQ ID NO: 8 - protein, 122 aa, Artificial [sequence omitted]

SEQ ID NO: 9 - protein, 119 aa, Artificial
  human consensus sequence of subgroup III
EVQLVESGGGLVQPGGSLRLSCAASGFTFSSYAMSWVRQAPGKGLEWVAVISGDGGSTYYADSVKGRFTISRDNSKNTLYLQMNSLRAEDTAVYYCARGRVGYSLYDYWGQGTLVTVSS

SEQ ID NO: 10 - protein, 10 aa, Artificial
  Heavy chain CDR1
GYTFTSYNMH

SEQ ID NO: 11 - protein, 17 aa, Artificial
  Heavy chain CDR2
AIYPGNGDTSYNQKFKG

SEQ ID NO: 12 - protein, 13 aa, Artificial
  Heavy chain CDR3
VVYYSNSYWYFDV

SEQ ID NO: 13 - protein, 213 aa, Artificial
  humanized 2H7 light chain sequence
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 452 aa, Artificial
  humanized 2H7 heavy chain sequence
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 452 aa, Artificial
  Humanized 2H7 heavy chain sequence
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 17 - protein, 309 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 18 - protein, 17 aa, Artificial
  BAFF antagonist
XCXDXLXXXXXXCXXXX

SEQ ID NO: 19 - protein, 17 aa, Artificial
  Formula 1 sequence
ECFDLLVRAWVPCSVLK

SEQ ID NO: 20 - protein, 17 aa, Artificial sequence
  Formula 1 sequence
ECFDLLVRHWVPCGLLR

SEQ ID NO: 21 - protein, 17 aa, Artificial
  Formula 1 sequence
ECFDLLVRRWVPCEMLG

SEQ ID NO: 22 - protein, 17 aa, Artificial
  Formula 1 sequence
ECFDLLVRSWVPCHMLR

SEQ ID NO: 23 - protein, 17 aa, Artificial [sequence omitted]

SEQ ID NO: 24 - protein, 16 aa, Artificial
  Formula 1 sequence
QCFDRLNAWVPCSVLK

SEQ ID NO: 25 - protein, 17 aa, Artificial
  BAFF antagonist Formula II
XCXDXLVXXWVPCXXLX

SEQ ID NO: 26 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 27 - protein, 26 aa, Artificial
  Mini-BR3
TPCVPAECFDLLVRHCVACGLLRTPR

SEQ ID NO: 28 - protein, 213 aa, Artificial
  hu2H7.v138 light chain region
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 452 aa, Artificial
  hu2H7.v138 heavy chain region
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 107 aa, Artificial
  Humanized 2H7 antibody light-chain variable region
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 31 - protein, 213 aa, Artificial; identical to SEQ ID NO: 28
  2H7.v511 light chain region

SEQ ID NO: 32 - protein, 452 aa, Artificial
  2H7.v511 heavy chain region
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK